(19) United States
(12) Patent Application Publication
(10) Pub. No.: US 2023/0279398 A1
(43) Pub. Date: Sep. 7, 2023

(54) TREATING HUMAN T-CELL LEUKEMIA VIRUS BY GENE EDITING

(71) Applicant: TEMPLE UNIVERSITY - OF THE COMMONWEALTH SYSTEM OF HIGHTER EDUCATION, Philadelphia, PA (US)

(72) Inventors: KAMEL KHALILI, Bala Cynyd, PA (US); HASSEN S. WOLLEBO, Philadelphia, PA (US)

(21) Appl. No.: 17/822,503

(22) Filed: Aug. 26, 2022

Related U.S. Application Data
(63) Continuation of application No. PCT/US2021/019909, filed on Feb. 26, 2021.
(60) Provisional application No. 62/982,156, filed on Feb. 27, 2020.

(51) Int. Cl. C12N 15/113; C12N 15/86

(52) U.S. Cl. CPC C12N 15/1132; C12N 15/86; C12N 2310/20

(57) ABSTRACT

Compositions which specifically target Human T cell leukemia virus (HTLV) coding sequences and other essential protein sequences, induce mutations and/or deletions in the viral DNA, rendering the virus unable to undergo replication and less likely to infect other cells, thus halting the viral life cycle and viral propagation and halting cellular transformation induced by the virus.

### CROSS-REFERENCE TO RELATED APPLICATIONS

This application is a continuation of International Application No. PCT/US2021/019909, filed Feb. 26, 2021, which claims the benefit of priority under 35 U.S.C. § 119(e) to U.S. Provisional Application No. 62/982,156, filed on Feb. 27, 2020, each of which are incorporated herein by reference in their entirety.

### INCORPORATION BY REFERENCE OF SEQUENCE LISTING

The contents of the sequence listing file named “348382.04301.xml”, which was created on Apr. 25, 2023 and is 9,293 bytes in size, are incorporated herein by reference in its entirety.

### FIELD OF THE DISCLOSURE

The present disclosure relates in general to compositions and methods of treating or eradicating Human T cell leukemia virus (HTL V) infections. The disclosure relates in particular to targeting of HTLV nucleic acid sequences by gene editing complexes.

### BACKGROUND

Human T cell leukemia virus type 1 (HTLV-1) was the first discovered human retrovirus and the etiologic agent of adult T-cell leukemia and FITLY-1-associated myelopathy/tropical spastic paraparesis. In the years since, several HTLV subtypes have been discovered: HTLV-2 was first identified in a patient with hairy cell leukemia, while HTLV-3 and HTLV-4 were discovered hi bushmeat hunters in Africa. HTLV is a zoonotic virus with simian T-cell leukemia virus counterparts found in monkeys. HTLV-1 and HTLV-2 are the most well studied subtypes of HTLV. They share roughly 70% nucleotide similarity and have a similar genome structure. Both viruses encode the structural and enzymatic proteins shared by all retroviruses, both encode the regulatory proteins Tax and Rex, and both feature a RNA transcript and protein derived from the negative-sense strand of the viral genome. HTLV-1 and HTLV-2 also express several accessory proteins that support various aspects of virus biology. HTLV-1 is associated with several diseases, including adult T-cell leukemia (ATL) and HTLV-1 associated myelopathy/tropical spastic paraparesis (HAM/TSP). There are an estimated five to ten million individuals infected with HTLV-1 worldwide with endemic regions of infection in Southwest Japan, sub-Saharan Africa, South America, the Caribbean, and regions of the Middle East and Australo-Melanesia. HTLV-1 demonstrates robust genetic stability. Mapping of stable nucleotide substitutions specific to varied geographic regions has been used to classify virus strains into geographic subtypes. The major geographic subtypes are Cosmopolitan subtype A, Central African subtype B, Australo-Melanesian subtype C, and Central African/Pygmies subtype D. Cosmopolitan subtype A is the most widespread subtype (endemic subgroups in Japan, Central and South America, the Caribbean, North and West Africa, and regions of the Middle East). Central African subtypes E, F, and G exist, but are rare.

### SUMMARY

Embodiments are directed to gene-editing complexes which specifically target the Human T cell leukemia virus (HTLV) genome. In certain embodiments, the complexes target at least two regions of the HTLV genome, e.g. HTLV-1 genome, whereby the genome between the two target regions is excised.

Also provided in various embodiments herein are various component parts of such a gene-editing complex, compositions comprising such a gene-editing complex (or one or more component part thereof), a vector comprising one or more nucleic acid sequence encoding one or more component of the gene editing complex (e.g., gRNA(s) and/or CRISPR protein(s) thereof, such as described in more detail herein). Also provided herein are methods of using the same, such as in excising part (e.g., about 50% or more, about 60% or more, about 70% or more, and/or a sufficient portion to inactivate the HTLV genome) or all of (or otherwise inactivating) the HTLV genome (e.g., from a host cell genome).

In some embodiments, a vector, nucleic acid, complex, or other composition provided herein excises or is capable of or suitable for excising part or all of an HTLV genome from a host cell genome or host cell, such as when provided to a host cell. In certain embodiments, at least about 50% or more, about 60% or more, about 70% or more, and/or a sufficient portion to inactivate the HTLV genome is excised from the host cell and/or genome thereof. In some embodiments, at least 100 nucleic acid base pair (bp), such as at least 150 bp, at least 200 bp, at least 250 bp, or at least 300 bp (of the HTLV genome) is excised from the host cell and/or genome thereof. In specific embodiments, at least 200 bp are excised. In more specific embodiments, at least 300 bp are excised.

In certain embodiments, the gene editing complex (or composition) comprises protein/nucleic acid or viral vector encoding the protein and/or nucleic acid (e.g., gRNA) which (e.g., specifically) targets Human T cell leukemia virus coding sequences and other essential protein sequences and induce mutations and/or deletions in the viral DNA, rendering the virus unable to undergo replication. This results in inhibiting infection of other cells, thus halting the viral life cycle and viral propagation and halting cellular transformation induced by the virus. In certain embodiments, the complexes target at least two regions of the HTLV genome whereby the genome between the two target regions is excised or mutated.

In some embodiments, a guide nucleic acid (e.g., gRNA) (such as in a complex provided herein and/or produced by a vector provided herein) targets (e.g., is complementary or hybridizes to) any suitable region of a HTLV genome. In certain embodiments, the complexes comprise one or more guide RNAs (gRNAs) which target early region nucleic acid sequences, late region nucleic acid sequences or combinations thereof.

In certain embodiments, a gene editing complex comprising an isolated nucleic acid sequence encoding a clustered regularly interspaced short palindromic repeats (CRISPR)-associated endonuclease and at least one guide RNA (gRNA), the gRNA being complementary to a target nucleic acid sequence in an Human T cell leukemia virus type 1 or 2 (HTLV-1 or -2) genome. In certain embodiments, the isolated nucleic acid sequences comprise a multiplex of gRNAs. In certain embodiments, the target nucleic acid sequence comprises one or more nucleic acid sequences in coding and non-coding nucleic add sequences of the HTLV-1 or HTLV-2 genome. In certain embodiments, the guide nucleic sequences comprise one or more guide RNAs (gRNAs) which target nucleic acid sequences comprising: LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof.

In certain embodiments, the guide nucleic acid sequence comprises a sequence comprising at least about 90% sequence identity to any one of SEQ ID NOS: 3-6, or a complement of any one of SEQ ID NOS: 3-6. In certain embodiments, the guide nucleic acid sequence comprises a sequence of any one of SEQ ID NOS: 3-6, or a complement of any one of SEQ ID NOS: 3-6 or combinations thereof. In certain embodiments, the target nucleic acid sequence comprises a sequence comprising at least about 90% sequence identity to any one of SEQ ID NOS: 3-6, or a complement of any one of SEQ ID NOS: 3-6. In certain embodiments, the target nucleic acid sequence comprises a sequence of any one of SEQ ID NOS: 3-6, or a complement of any one of SEQ ID NOS: 3-6 or combinations thereof. In certain embodiments, the target nucleic acid sequences comprise a sequence comprising at least about 90% sequence identity to at least five consecutive nucleotides of SEQ ID NOS: 1 or 2, or a complement of at least five consecutive nucleotides of SEQ II) NOS: 1 or 2. In certain embodiments, the target nucleic acid sequence comprises at least five consecutive nucleotides of SEQ ID NOS: 1 or 2, or at least five consecutive nucleotides complementary to SEQ ID NOS: 1 or 2, or combinations thereof.

In certain embodiments, a composition comprises an isolated nucleic acid sequence encoding a clustered regularly interspaced short palindromic repeats (CRISPR)-associated endonuclease and at least two guide nucleic acid sequences, each guide nucleic acid sequence being complementary to a target nucleic acid sequence in a Human T cell leukemia virus type 1 or 2 (HTLV-1 or -2) genome. In certain embodiments, the target nucleic acid sequence comprises one or more nucleic acid sequences in coding and non-coding nucleic acid sequences of the HTLV-1 or HTLV-2 genome. In certain embodiments, the isolated nucleic acid sequences are included in at least one expression vector selected from the group consisting of: a lentiviral vector, an adenovirus vector, an adeno-associated virus vector, a vesicular stomatitis virus (VSV) vector, a pox virus vector, and a retroviral vector. In certain embodiments, the guide nucleic sequences comprise one or more guide RNAs (gRSAs) which target nucleic acid sequences comprising one or more of: LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof. In certain embodiments, the one or more guide RNAs (gRNAs) complementary to any one or more nucleic acid sequences comprise LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof.

In certain embodiments, a composition comprises: a) a Clustered Regularly Interspaced Short Palindromic Repeat (CRISPR)-associated endonuclease or a nucleic acid sequence encoding the CRISPR-associated endonuclease; b) one or more guide nucleic acids, wherein the guide nucleic acids comprise nucleotide sequences substantially complementary to a target sequence comprising: LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof.

In certain embodiments, a composition comprises a) a Clustered Regularly Interspaced Short Palindromic Repeat (CRISPR)-associated endonuclease or a nucleic acid sequence encoding the CRISPR-associated endonuclease; b) one or more guide nucleic acids, wherein the guide nucleic acids comprise nucleotide sequences substantially complementary to a target sequence comprising: LTR nucleic acid sequences, Gag nucleic acid sequences, Pol nucleic acid sequences, Pro nucleic acid sequences, Env nucleic acid sequences, pX region nucleic acid sequences, HIV nucleic acid sequences, APH-2 nucleic acid sequences, Tax-1 nucleic acid sequences, Tax-2 nucleic acid sequences or combinations thereof.

In certain embodiments, a composition comprises a) a Clustered Regularly interspaced Short Palindromic Repeat (CRISPR)-associated endonuclease or a nucleic acid sequence encoding the CRISPR-associated endonuclease; b) a first guide nucleic acid or a nucleic acid sequence encoding the first guide nucleic acid, the first guide nucleic acid being complementary to a first target nucleic acid sequence within a 5′- and/or 3′-long terminal repeat (LTR) gene; c) a second guide nucleic acid or a nucleic acid sequence encoding the second guide nucleic acid, the second guide nucleic acid being complementary to a second target nucleic acid sequence within a Gag gene. In certain embodiments, the composition further comprises a third guide nucleic acid or a nucleic acid sequence encoding the guide nucleic acid, the third guide nucleic acid being complementary to a third target nucleic acid sequence within a Pol gene. In certain embodiments, the composition further comprises a fourth guide nucleic acid or a nucleic acid sequence encoding the guide nucleic acid, the fourth guide nucleic acid being complementary to a fourth target nucleic acid sequence within a Pro gene. In certain embodiments, the composition further comprises a fifth guide nucleic acid or a nucleic acid sequence encoding the guide nucleic, acid, the fifth guide nucleic acid being complementary to a fifth target nucleic acid sequence within an Env gene. In certain embodiments, the composition further comprises a sixth guide nucleic acid or a nucleic acid sequence encoding the guide nucleic acid, the sixth guide nucleic acid being complementary to a sixth target nucleic acid sequence within a pX region gene. In certain embodiments, the composition further comprises a seventh guide nucleic acid or a nucleic acid sequence encoding the guide nucleic acid, the seventh guide nucleic acid being complementary to a seventh target nucleic acid sequence within an HIM gene. In certain embodiments, the composition further comprises an eighth guide nucleic acid or a nucleic acid sequence encoding the guide nucleic acid, the eighth guide nucleic acid being complementary to an eighth target nucleic acid sequence within an APH-2 gene. In certain embodiments, the composition further comprises a ninth guide nucleic acid or a nucleic acid sequence encoding the guide nucleic acid, the ninth guide nucleic acid being complementary to a ninth target nucleic acid sequence within a Tax-1 gene. In certain embodiments, the composition further comprises a tenth guide nucleic acid or a nucleic acid sequence encoding the guide nucleic acid, the tenth guide nucleic acid being complementary to a tenth target nucleic acid sequence within a Tax-2 gene.

In certain embodiments, a composition comprises: a) a Clustered Regularly Interspaced Short Palindromic Repeat (CRISPR)-associated endonuclease or a nucleic acid sequence encoding the CRISPR-associated endonuclease; b) at least two guide nucleic acids or nucleic acid sequences encoding: i. a first guide nucleic acid, the first guide nucleic acid being complementary to a first target nucleic acid sequence within a 5′-LTR gene; ii. a second guide nucleic acid, the second guide nucleic acid being complementary to a second target nucleic acid sequence within a 3′-LTR gene; wherein the first target nucleic acid sequence and the second target nucleic acid sequence, are different.

In certain embodiments, a composition comprises: a) a Clustered Regularly Interspaced Short Palindromic Repeat (CRISPR)-associated endonuclease or a nucleic acid sequence encoding the CRISPR-associated endonuclease; b) at least two guide nucleic acids or nucleic acid sequences encoding: a first guide nucleic acid, the first guide nucleic acid being complementary to a first target nucleic acid sequence within a Gag gene; a second guide nucleic acid, the second guide nucleic acid being complementary to a second target nucleic acid sequence within a Gag gene; wherein the first target nucleic acid sequence and the second target nucleic acid sequence, are different.

In certain embodiments, a composition comprises: a) a Clustered Regularly Interspaced Short Palindromic Repeat (CRISPR)-associated endonuclease or a nucleic acid sequence encoding the CRISPR-associated endonuclease; b) at least two guide nucleic acids or nucleic acid sequences encoding: a first guide nucleic acid, the first guide nucleic acid being complementary to a first target nucleic acid sequence within a Poll gene; a second guide nucleic acid, the second guide nucleic acid being complementary to a second target nucleic acid sequence within a Pol gene; wherein the first target nucleic acid, sequence and the second target nucleic acid sequence, are different.

In certain embodiments, a composition comprises: a) a Clustered Regularly Interspaced Short Palindromic Repeat (CRISPR)-associated endonuclease or a nucleic acid sequence encoding the CRISPR-associated endonuclease; b) at least two guide nucleic acids or nucleic acid sequences encoding: a first guide nucleic acid, the first guide nucleic acid being complementary to a first target nucleic acid sequence within a Pro gene; a second guide nucleic acid, the second guide nucleic acid being complementary to a second target nucleic acid sequence within a Pro gene; wherein the first target nucleic acid sequence and the second target nucleic acid sequence, are different.

In certain embodiments, a composition comprises: a) a Clustered Regularly Interspaced Short Palindromic Repeat (CRISPR)-associated endonuclease or a nucleic acid sequence encoding the CRISPR-associated endonuclease; b) at least two guide nucleic acids or nucleic acid sequences encoding: a first guide nucleic acid, the first guide nucleic acid being complementary to a first target nucleic acid sequence within an Env gene; a second guide nucleic acid, the second guide nucleic acid being complementary to a second target nucleic acid sequence within an Env gene; wherein the first target nucleic acid sequence and the second target nucleic acid sequence, are different.

In certain embodiments, a composition comprises: a) a Clustered Regularly Interspaced Short Palindromic Repeat (CRISPR)-associated endonuclease or a nucleic acid sequence encoding the CRISPR-associated endonuclease; b) at least two guide nucleic acids or nucleic acid sequences encoding: a first guide nucleic acid, the first guide nucleic acid being complementary to a first target nucleic acid sequence within a pX region gene; a second guide nucleic acid, the second guide nucleic acid being complementary to a second target nucleic acid sequence within a pX region gene; wherein the first target nucleic acid sequence and the second target nucleic acid sequence, are different.

In certain embodiments, a composition comprises: a) a Clustered Regularly Interspaced Short Palindromic Repeat (CRISPR)-associated endonuclease or a nucleic acid sequence encoding the CRISPR-associated endonuclease; b) at least two guide nucleic acids or nucleic acid sequences encoding: a first guide nucleic acid, the first guide nucleic acid being complementary to a first target nucleic acid sequence within an HBZ gene; a second guide nucleic acid, the second guide nucleic acid being complementary to a second target nucleic acid sequence within an HBZ gene; wherein the first target nucleic acid sequence and the second target nucleic acid sequence, are different.

In certain embodiments, a composition comprises: a) a Clustered Regularly Interspaced Short Palindromic Repeat (CRISPR)-associated endonuclease or a nucleic acid sequence encoding the CRISPR-associated endonuclease; b) at least two guide nucleic acids or nucleic acid sequences encoding: a first guide nucleic acid, the first guide nucleic acid being complementary to a first target nucleic acid sequence within an APH-2 gene; a second guide nucleic acid, the second guide nucleic acid being complementary to a second target nucleic acid sequence within an APH-2 gene; wherein the first target nucleic acid sequence and the second target nucleic acid sequence, are different.

In certain embodiments, a composition comprises: a) a Clustered Regularly Interspaced Short Palindromic Repeat (CRISPR)-associated endonuclease or a nucleic acid sequence encoding the CRISPR-associated endonuclease; b) at least two guide nucleic acids or nucleic acid sequences encoding: a first guide nucleic acid, the first guide nucleic acid being complementary to a first target nucleic acid sequence within a Tax-1 gene; a second guide nucleic acid the second guide nucleic acid being complementary to a second target nucleic acid sequence within a Tax-1 gene; wherein the first target nucleic acid sequence and the second target nucleic acid sequence, are different.

In certain embodiments, a composition comprises: a) a Clustered Regularly Interspaced Short Palindromic Repeat (CRISPR)-associated endonuclease or a nucleic acid sequence encoding the CRISPR-associated endonuclease; b) at least two guide nucleic acids or nucleic acid sequences encoding: a first guide nucleic acid, the first guide nucleic acid being complementary to a first target nucleic acid sequence within a Tax-2 gene; a second guide nucleic acid, the second guide nucleic acid being complementary to a second target nucleic acid sequence within a Tax-2 gene; wherein the first target nucleic acid sequence and the second target nucleic acid sequence, are different.

In certain embodiments, a composition comprises: a) a Clustered Regularly Interspaced Short Palindromic Repeat (CRISPR)-associated endonuclease or a nucleic acid sequence encoding the CRISPR-associated endonuclease; b) a plurality of guide nucleic acids or nucleic acid sequences encoding one or more combinations of guide nucleic acids, comprising: two or more guide nucleic acids wherein each guide nucleic acid being complementary to two or more target nucleic acid sequences within an LTR gene, wherein each nucleic acid target sequence in the LTR gene is different; two or more guide nucleic acids wherein each guide nucleic acid being complementary to two or more target nucleic acid sequences within a Gag gene, wherein each nucleic acid target sequence in the Gag gene is different; two or more guide nucleic acids wherein each guide nucleic acid being complementary to two or more target nucleic acid sequences within a Pol gene, wherein each nucleic acid target sequence in the Pol gene is different; two or more guide nucleic acids wherein each guide nucleic acid being complementary to two or more target nucleic acid sequences within a Pro gene, wherein each nucleic acid target sequence in the alternate frame of the Pro gene is different; two or more guide nucleic acids wherein each guide nucleic acid being complementary to two or more target nucleic acid sequences within an Env gene, wherein each nucleic acid target sequence in the Env gene is different; two or more guide nucleic acids wherein each guide nucleic acid being complementary to two or more target nucleic acid sequences within a pX region gene, wherein each nucleic acid target sequence in the pX region gene is different; two or more guide nucleic acids wherein each guide nucleic acid being complementary to two or more target nucleic acid sequences within an HBZ gene, wherein each nucleic acid target sequence in the HBZ gene is different; two or more guide nucleic acids wherein each guide nucleic acid being complementary to two or more target nucleic acid sequences within an APH-2 gene, wherein each nucleic acid target sequence in the APH-2 gene is different; two or more guide nucleic acids wherein each guide nucleic acid being complementary to two or more target nucleic acid sequences within a Tax-1 gene, wherein each nucleic acid target sequence in the Tax-1 gene is different; two or more guide nucleic acids wherein each guide nucleic acid being complementary to two or more target nucleic acid sequences within a Tax-2 Rene, wherein each nucleic acid target sequence in the Tax-2 gene is different.

In certain embodiments, an expression vector comprises a nucleic acid encoding: a) a Clustered Regularly interspaced Short Palindromic Repeat (CRISPR)-associated endonuclease or a nucleic acid sequence encoding the CRISPR-associated endonuclease; b) one or a plurality of guide nucleic acids or nucleic acid sequences encoding one or more combinations of guide nucleic acids, comprising: two or more guide nucleic acids wherein each guide nucleic acid being complementary to two or more target nucleic acid sequences within an LTR gene, wherein each nucleic acid target sequence in the LTR gene is different, two or more guide nucleic acids wherein each guide nucleic acid being complementary to two or more target nucleic acid sequences within a Gag gene, wherein each nucleic acid target sequence in the Gag gene is different; two or more guide nucleic acids wherein each guide nucleic acid being complementary to two or more target nucleic acid sequences within a Pol gene, wherein each nucleic acid target sequence in the Pol gene is different; two or more guide nucleic acids wherein each guide nucleic acid being complementary to two or more target nucleic acid sequences within a Pro gene, wherein each nucleic acid target sequence in the alternate frame of the Pro gene is different; two or more guide nucleic acids wherein each guide nucleic acid being complementary to two or more target nucleic acid sequences within an Env gene, wherein each nucleic acid target sequence in the Env gene is different; two or more guide nucleic acids wherein each guide nucleic acid being complementary to two or more target nucleic acid sequences within a pX region gene, wherein each nucleic acid target sequence in the pX region gene is different; two or more guide nucleic acids wherein each guide nucleic acid being complementary to two or more target nucleic acid sequences within an HBZ gene, wherein each nucleic acid target sequence in the HBZ gene is different; two or more guide nucleic acids wherein each guide nucleic acid being complementary to two or more target nucleic acid sequences within an APH-2 gene, wherein each nucleic acid target sequence in the APH-2 gene is different; two or more guide nucleic acids wherein each guide nucleic acid being complementary to two or more target nucleic acid sequences within a Tax-1 gene, wherein each nucleic acid target sequence in the Tax-1 gene is different; two or more guide nucleic acids wherein each guide nucleic acid being complementary to two or more target nucleic acid sequences within a Tax-2 gene, wherein each nucleic acid target sequence in the Tax-2 gene is different.

In certain embodiments, the CRISPR-associated endonuclease is a Type I, Type II, or Type III Cas endonuclease. In certain embodiments, the CRISPR-associated endonuclease is a Cas9 endonuclease, a Cas12 endonuclease, a Cas 13 endonuclease, a CasX endonuclease, a CasΦ endonuclease or variants thereof. In certain embodiments, the CRISPR-associated endonuclease is a Cas9 nuclease or variants thereof. In certain embodiments, the Cas9 nuclease is a Staphylococcus aureus Cas9 nuclease. In certain embodiments, the Cas9 variant comprises one or more point mutations, relative to wildtype Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A. In certain embodiments, a Cas9 variant comprises a human-optimized Cas9; a nickase mutant Cas9; saCas9; enhanced-fidelity SaCas9 (efSaCas9); SpCas9(K855a); SpCas9(K810A/K1003A/r1060A); SpCas9(K848A/K1003A/R1060A); SpCas9 N497A, R661A, Q695A, Q926A, SpCas9 N497A, R661A, Q695A, Q926A, D1135E; SpCas9 N497A, R661A, Q695A, Q926A L169A; SpCas9 N497A, R661A, Q695A, Q926A Y450A; SpCas9 N497A, R661A, Q695A, Q926A M495A; SpCas9 N497A, R661A, Q695A, Q926A M694A; SpCas9 N497A, R661A, Q695A, Q926A H698A; SpCas9 N497A, R661A, Q695A, Q926A, D1135E, L169A; SpCas9 N497A, R661A, Q695A, Q926A, D1135E, Y450A; SpCas9 N497A, R661A, Q695A, Q926A, D1135E, M495A; SpCas9 N497A, R661A, Q695A, Q926A, D1135E, M694A; SpCas9 N497A, R661A, Q695A, Q926A, D1135E, M698A; SpCas9 R661A, Q695A, Q926A; SpCas9 R661A, Q695A, Q926A, D1135E; SpCas9 R661A, Q695A, Q926A, L169A; SpCas9 R661A, Q695A, Q926A Y450A, SpCas9 R661A, Q695A, Q926A M495A; SpCas9 R661A, Q695A, Q926A M694A SpCas9 R661A, Q695A, Q926A H698A; SpCas9 R661A, Q695A, Q926A D1135E L169A; SpCas9 R661A, Q695A, Q926A D1135E Y450A; SpCas9 R661A, Q695A, Q926A D1135E M495A; or SpCas9 R661A, Q695A, Q926A, D1135E or M694A. In certain embodiments, the CRISPR-associated endonuclease is optimized for expression in a human cell.

In certain embodiments, the isolated nucleic acid sequences are included in at least one expression vector selected from the group consisting of: a lentiviral vector, an adenovirus vector, an adeno-associated virus hector, a vesicular stomatitis virus (VSV) vector, a pox virus vector, and a retroviral vector. In certain embodiments, the expression vector comprises: a lentiviral vector, an adenoviral vector, or an adeno-associated virus vector. In certain embodiments, the adeno-associated virus (AAV) vector is AV1, AAV2, AAV3, AAV4, AAV5, AAV6, AAV7, AAV8, AAV9, AAV10, AAV11, AAVDJ, or AAVDJ/8. In certain embodiments, the vector comprising the nucleic acid further comprises a promoter. In certain embodiments, the promoter comprises a ubiquitous promoter, a tissue-specific promoter, an inducible promoter or a constitutive promoter.

In certain embodiments, a method of treating a subject infected with a Human T cell leukemia virus (HTLV) comprises administering to the subject an effective amount of the gene editing complexes compositions embodied herein, whereby the genome between the two target regions is excised.

Any compositions or methods provided herein can be combined with one or more of any of the other compositions and methods provided herein.

### Definitions

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are described.

As used herein, each of the following terms has the meaning associated with it in this section.

The articles “a” and “an” are used herein to refer to one or to more than one (i.e., to at least one) of the grammatical object of the article. By way of example. “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods. In specific instances, unless otherwise indicated, “about” is ±10%.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the seventy of a symptom of the disease or disorder, the frequency with which such a symptom is experienced by a patient, or both, is reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the an, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can gist in a non-native Environment such as, for example, a host cell.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introits to the extent that the nucleotide sequence encoding the protein may in some version contain an intron(s).

The terms “patient,” “subject,” “individual,” and the like are used interchangeably herein, and refer to any animal, or cells thereof whether in yarn or in situ, amenable to the methods described herein. In certain non-limiting embodiments, the patient, subject or individual is a human.

“Parenteral” administration of a composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art including, without limitation, recombinant means, i.e., the cloning of nucleic acid sequences from a recombinant library or a cell genome, using ordinary cloning technology and PCR™, and the like, and by synthetic means.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introits to the extent that the nucleotide sequence encoding the protein may in some version contain an intron(s).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. In embodiments, two sequences are 100% identical. In embodiments, two sequences are 100% identical over the entire length of one of the sequences (e.g., the shorter of the two sequences where the sequences have different lengths). In embodiments, identity may refer to the complement of a test sequence. In embodiments, the identity exists over a region that is at least about 10 to about 100, about 20 to about 75, about 30 to about 50 amino acids or nucleotides in length. In embodiments, the identity exists over a region that is at least about 50 amino acids or nucleotides in length, or more over a region that is 100 to 500, 100 to 200, 150 to 200, 175 to 200, 175 to 225, 175 to 250, 200 to 225, 200 to 250 or more amino acids or nucleotides in length.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutical) acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

As used in this specification and the appended claims, the term “or” is generally employed in its sense including “and/or” unless the content clearly dictates otherwise.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “therapeutic treatment” is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

To “treat” a disease as the term is used herein, means to reduce the frequency or severity of at least one sign or symptom of a disease or disorder experienced by a subject.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### BRIEF DESCRIPTION OF THE DRAWINGS

FIG. 1 is a series of blots demonstrating the excision of HTLV-1 based on the sequences shown in the figure.

HTLV-1 full length sequence (SEQ ID NO: 1):
CCACTCTAACCTAGACCATATCCTCG/94/ATCCAC
TTGGCACGTCCTATACTCTC . . . /1,720/ . . . C
AAATACTCCCCCTTCCGAAATGGAT/59/CGGCCCCAAAACC
TGTACACCCTCT
HTLV-1 Excision (SEQ ID NO: 2):
CCACTCTAACCTAGACCATATCCTCG/94/ATCCACTTGGCA
CGTCCTATACTCTCCCA/122/GTC. . . /
1,545/ . . . ACT/
41/GCGCAAATACTCCCCCTTCCGAAATGGA
T/59/CGGCCCCAAAACCTGTACACCCTCT

### Guide Nucleic Acid Sequences

SEQ ID NO: 3
ATCCACTTGGCACGTCCTATACTCTC
SEQ ID NO. 4
CAAATACTCCCCCTTCCGAAATGGAT
SEQ ID NO: 5
ATCCACTTGGCACGTCCTATACTCTCCCA
SEQ ID NO: 6
GCGCAAATACTCCCCCTTCCGAAATGGAT

Bolded nucleotides are the PAM sequences.

FIG. 2 is a series of blots demonstrating the excision of HTLV-1 based on the sequences shown in the figure.

HTLV-1 full length sequence (SEQ ID NO: 1):
CCACTCTAACCTAGACCATATCCTCG/94/ATCCAC
TTGGCACGTCCTATACTCTC . . . /1,720/ . . . C
AAATACTCCCCCTTCCGAAATGGAT/59/CGGCCCCAAAACC
TGTACACCCTCT
HTLV-1 Excision (SEQ ID NO: 2):
CCACTCTAACCTAGACCATATCCTCG/94/ATCCACTTGGCA
CGTCCTATACTCTCCCA/122/GTC. . . /
1,545/ . . . ACT/
41/GCGCAAATACTCCCCCTTCCGAAATGGA
T/59/CGGCCCCAAAACCTGTACACCCTCT

### Guide Nucleic Acid Sequences

SEQ ID NO: 3
ATCCACTTGGCACGTCCTATACTCTC
SEQ ID NO. 4
CAAATACTCCCCCTTCCGAAATGGAT
SEQ ID NO: 5
ATCCACTTGGCACGTCCTATACTCTCCCA
SEQ ID NO: 6
GCGCAAATACTCCCCCTTCCGAAATGGAT

Bolded nucleotides are the PAM sequences.

### DETAILED DESCRIPTION

Human T cell leukemia virus type 1 or 2 (HTLV-1 or -2) are members of the delta retrovirus family. These viruses are complex retroviruses that express regulatory and accessory genes, in addition to the structural and enzymatic genes common to all retroviruses. The proviral genomes of HTLV-1 and HTLV-2 are roughly 9 kb in length and feature 4′ and 3′ long terminal repeats (LTR), which are direct repeats generated during the reverse transcription process. The 5′ portions of both genomes encode the structural and enzymatic gene products (Gag, Pol, Pro, and Env). The regulatory and accessory genes are expressed from the historically termed ‘pX’ region of the genome. The pX region is located 3′ of the structural gene Env. Both HTLVs encode an antisense gene, HBZ for HTLV-1 and APH-2 for HTLV-2, located on the negative or minus strand of the proviral genome.

HTLV-1 and HTLV-2 encode the pleiotropic transactivator proteins Tax-1 and Tax-2, respectively, which share 85% amino acid identity. Both proteins contain CREB-activating domains (N-termini), zinc finger domains (N-termini), nuclear localization signals (Tax-1, within first 60 amino acids; Tax-2, within first 42 amino acids), nuclear export signals (amino acids 189-202) and ATP/CREB activating domains (C-termini regions). Unlike Tax-2, Tax-1 has two leucine upper-like regions (amino acids 116-145 and 225-232) responsible for activation of the canonical and non-canonical NF-κB pathways, a PDZ-binding motif (PBM; C-terminal 4 amino acids), and a secretory signal (C-terminus). Conversely, Tax-2 has a cytoplasmic localization domain (amino acids 89-113), which Tax-1 lacks. Although Tax-1 and Tax-2 have been found in both the nuclear and cytoplasmic compartments of infected cells, the Tax-2 cytoplasmic localization domain explains its primarily cytoplasmic distribution when compared to the primarily nuclear distribution of Tax-1. Despite their functional domain similarities, the Tax-1 and Tax-2 interactomes and subsequent effects on cellular pathways are divergent.

The integrated retroviral genome has two identical copies of long terminal repeat (LTR). Targeting the LTR is advantageous because the number of therapeutic targets per provirus is doubled.

In certain embodiments, a method of preventing or treating an HTLV infection in a subject, comprising: administering to the subject a pharmaceutical composition comprising a therapeutically effective amount of an isolated nucleic acid sequence encoding a Clustered Regularly Interspaced Short Palindromic Repeat (CRISPR)-associated endonuclease and at least one guide RNA (gRNA), the gRNA being complementary to a target sequence in the integrated retroviral DNA.

Combinations of gRNAs are especially effective when expressed in multiplex fashion, that is, simultaneously in the same cell.

In certain embodiments, the at least one gRNA includes at least a first gRNA that is complementary to a target sequence in the integrated retroviral DNA; and a second gRNA that is complementary to another target sequence in the integrated retroviral DNA, whereby the intervening sequences between the two gRNAs are excised.

In certain embodiments, the gene editing complex comprises a first gRNA that is complementary to a target sequence in the integrated retroviral DNA; and a second gRNA that is complementary to another target sequence in the integrated retroviral DNA, whereby the intervening sequences between the two gRNAs are excised.

In embodiments, the compositions of the invention include nucleic acids encoding gene editing agents and at least one guide RNA (gRNA) that is complementary to a target sequence in a retrovirus, e.g. HTLV. In embodiments, the gene editing agents comprise: Cre recombinases, CRISPR/Cas molecules, TALE transcriptional activators, Cas9 nucleases, nickases, transcriptional regulators, homologues, orthologs or combinations thereof.

Provided herein, in some embodiments, are methods and compositions comprising a CRISPR-associated (Cas) peptide or a nucleic acid sequence encoding the CRISPR-associated (Cas) peptide and a plurality of guide nucleic acids or a nucleic acid sequence encoding the plurality of guide nucleic acids. In some embodiments, compositions and methods described herein comprise 1, 2, 3, 4, 5, 6, or more than 6 gRNAs. In some embodiments, compositions and methods described herein comprise 1, 2, 3, 4, 5, 6, or more than 6 different gRNAs. In some embodiments, compositions and methods described herein comprise 4 or at least 4 different gRNAs.

In certain embodiments, the compositions of the disclosure include nucleic acids encoding gene editing agents and at least one guide RNA (gRNA) that is complementary to a target sequence in a retrovirus, e.g. Human T cell leukemia virus type 1 or 2 (HTLV-1 or -2). In certain embodiments, the gene editing agents comprise: Cre recombinases, CRISPR/Cas molecules, TALE transcriptional activators, Cas9 nucleases, nickases, transcriptional regulators, homologues, orthologs or combinations thereof. In certain embodiments, the retrovirus target sequences comprise coding sequences, noncoding sequences or combinations thereof. In certain embodiments, the guide nucleic acid sequences target one or more HTLV sequences comprising: structural gene sequences, enzymatic gene sequences, regulatory genes, accessory genes, transactivator gene sequences or combinations thereof. In certain embodiments, HTLV target sequences comprise sequences in the LTR, Gag, Pol, Pro, Env, pX region, HBZ, APH-2, Tax-1, Tax-2 or combinations thereof.

In one embodiment, a composition comprises a viral vector encoding a gene editing agent and at least one guide RNA (gRNA) wherein the gRNA is complementary to a target nucleic acid sequence of an HTLV gene sequence, comprising: a target nucleic acid sequence of a coding and/or non-coding HTLV gene sequence. In this embodiment, the gene editing agent is a Clustered Regularly Interspaced Short Palindromic Repeated (CRISPR)-associated endonuclease, or homologues thereof. An example of a CRISPR associated endonuclease is Cas9 or homologues or orthologs thereof.

In another embodiment, an expression vector comprises an isolated nucleic acid encoding a gene editing agent and at least one guide RNA (gRNA) wherein the gRNA is complementary to a target nucleic acid sequence of a retrovirus, e.g. HTLV-1, HTLV-2 gene sequences. In embodiments, the gene editing agents comprise. Cre recombinases, CRISPR/Cas molecules, TALE transcriptional activators, Cas9 nucleases, nickases, transcriptional regulators, homologues, orthologs or combinations thereof. In one embodiment, the gene editing agent is a Clustered Regularly Interspaced Short Palindromic Repeated (CRISPR)-associated endonuclease, or homologues or orthologs thereof. In another embodiment, the CRISPR-associated endonuclease is Cas9 or homologues or ortholog, thereof.

Provided herein, in certain embodiments, are methods and compositions for targeting the LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof, using at least one guide nucleic acid or a plurality of guide nucleic acids.

In some embodiments, different gRNAs target different sequences within the 5′-and/or 3′ long terminal repeat (LTR) genes. In some embodiments, the different gRNAs are complementary to different target sequences within the LTR gene. In some embodiments, a target sequence is within or near the LTR gene. In some embodiments, a region near the LTR gene comprises 1, 2, 3, 4, 5, 10, 15, 20, 25, 30, or 35 base positions surrounding the LTR gene.

In some embodiments, the different gRNAs target different sequences within the Gag gene. In some embodiments, the different gRNAs are complementary to different target sequences within the Gag gene. In some embodiments, a target sequence is within or near the Gag gene. In some embodiments, a region near the Gag gene comprises 1, 2, 3, 4, 5, 10, 15, 20, 25, 30, or 35 base positions surrounding the Gag gene.

In some embodiments, the different gRNAs target different sequences within the Pol gene. In some embodiments, the different gRNAs are complementary to different target sequences within the Pol gene. In some embodiments, a target sequence is within or near the Pol gene. In some embodiments, a region near the Pol gene comprises 1, 2, 3, 4, 5, 10, 15, 20, 25, 30, or 35 base positions surrounding the Pol gene.

In some embodiments, the different gRNAs target different sequences within the Pro gene. In some embodiments, the different gRNAs are complementary to different target sequences within the Pro gene. In some embodiments, a target sequence is within or near the Pro gene. In some embodiments, a region near the Pro gene comprises 1, 2, 3, 4, 5, 10, 15, 20, 25, 30, or 35 base positions surrounding the Pro gene.

In some embodiments, the different gRNAs target different sequences within the Env gene. In some embodiments, the different gRNAs are complementary to different target sequences within the Env gene. In some embodiments, a target sequence is within or near the Env gene. In some embodiments, a region near the Env gene comprises 1, 2, 3, 4, 5, 10, 15, 20, 25, 30, or 35 base positions surrounding the Env gene.

In some embodiments, the different gRNAs target different sequences within the pX region gene. In some embodiments, the different gRNAs are complementary to different target sequences within the pX region gene. In some embodiments, a target sequence is within or near the pX region gene. In some embodiments, a region near the pX region gene comprises 1, 2, 3, 4, 5, 10, 13, 20, 25, 30, or 35 base positions surrounding the pX region gene.

In some embodiments, the different gRNAs target different sequences within the HBZ or APH-2 genes. In some embodiments, the different gRNAs are complementary to different target sequences within the HBZ or APH-2 genes. In some embodiments, a target sequence is within or near the HBZ or APH-2 genes. In some embodiments, a region near the HBZ or APH-2 genes comprises 1, 2, 3, 4, 5, 10, 15, 20, 25, 30, or 35 base positions surrounding the HBZ or APH-2 genes.

In some embodiments, the different gRNAs target different sequences within the Tax-1 gene. In some embodiments, the different gRNAs are complementary to different target sequences within the Tax-1 gene. In some embodiments, a target sequence is within or near the Tax-1 gene. In some embodiments, a region near the Tax-1 gene comprises 1, 2, 3, 4, 5, 10, 15, 20, 25, 30, or 35 base positions surrounding the Tax-1 gene.

In some embodiments, the different gRNAs target different sequences within the Tax-2 gene. In some embodiments, the different gRNAs are complementary to different target sequences within the Tax-2 gene. In some embodiments, a target sequence is within or near the Tax-2 gene. In some embodiments, a region near the Tax-2 gene comprises 1, 2, 3, 4, 5, 10, 15, 20, 25, 30, or 35 base positions surrounding the Tax-2 gene.

In some embodiments, a first guide nucleic acid of the plurality of guide nucleic acids is complementary to a first target sequence in an LTR gene. In some embodiments, a second guide nucleic acid of the plurality of guide nucleic acids is complementary to a second target sequence in an LTR gene. In some embodiments, a third guide nucleic acid of the plurality of guide nucleic acid is complementary to a third target sequence in an LTR gene. In some embodiments, a fourth guide nucleic acid of the plurality of guide nucleic acid is complementary to a fourth target sequence in an LTR gene. In some embodiments, the first target sequence, the second target sequence, the third target sequence, and the fourth target sequence are different, wherein the intervening sequences between pairs of guide nucleic acids are excised or inactivate the expression or function of the LTR gene.

In some embodiments, a first guide nucleic acid of the plurality of guide nucleic acids is complementary to a first target sequence in a Gag gene. In some embodiments, a second guide nucleic acid of the plurality of guide nucleic acids is complementary to a second target sequence in a Gag gene. In some embodiments, a third guide nucleic acid of the plurality of guide nucleic acid is complementary to a third target sequence in a Gag gene. In some embodiments, a fourth guide nucleic acid of the plurality of guide nucleic acid is complementary to a fourth target sequence in a Gag gene. In some embodiments, the first target sequence, the second target sequence, the third target sequence, and the fourth target sequence are different, wherein the intervening sequences between pairs of guide nucleic acids are excised or inactivate the expression or function of the Gag gene.

In some embodiments, a first guide nucleic acid of the plurality of guide nucleic acids is complementary to a first target sequence in a Pol gene. In some embodiments, a second guide nucleic acid of the plurality of guide nucleic acids is complementary to a second target sequence in a Pol gene. In some embodiments, a third guide nucleic acid of the plurality of guide nucleic acid is complementary to a third target sequence in a Pol gene. In some embodiments, a fourth guide nucleic acid of the plurality of guide nucleic acid is complementary to a fourth target sequence in a Pol gene. In some embodiments, the first target sequence, the second target sequence, the third target sequence, and the fourth target sequence are different, wherein the intervening sequences between pairs of guide nucleic acids are excised or inactivate the expression or function of the Pol gene.

In some embodiments, a first guide nucleic acid of the plurality of guide nucleic acids is complementary to a first target sequence in a Pro gene. In some embodiments, a second guide nucleic acid of the plurality of guide nucleic acids is complementary to a second target sequence in a Pro gene. In some embodiments, a third guide nucleic acid of the plurality of guide nucleic acid is complementary to a third target sequence in a Pro gene. In some embodiments, a fourth guide nucleic acid of the plurality of guide nucleic acid is complementary to a fourth target sequence in a Pro gene. In some embodiments, the first target sequence, the second target sequence, the third target sequence, and the fourth target sequence are different wherein the intervening sequences between pairs of guide nucleic acids are excised or inactivate the expression or function of the Pro gene.

In some embodiments, a first guide nucleic acid of the plurality of guide nucleic acids is complementary to a first target sequence in a Env gene. In some embodiments, a second guide nucleic acid of the plurality of guide nucleic acids is complementary to a second target sequence in a Env gene. In some embodiments, a third guide nucleic acid of the plurality of guide nucleic acid is complementary to a third target sequence in a Env gene. In some embodiments, a fourth guide nucleic acid of the plurality of guide nucleic acid is complementary to a fourth target sequence in a Env gene. In some embodiments, the first target sequence, the second target sequence, the third target sequence, and the fourth target sequence are different, wherein the intervening sequences between pairs of guide nucleic acids are excised or inactivate the expression or function of the Env gene.

In some embodiments, a first guide nucleic acid of the plurality of guide nucleic acids is complementary to a first target sequence in a pX region gene. In some embodiments, a second guide nucleic acid of the plurality of guide nucleic acids is complementary to a second target sequence in a pX region gene. In some embodiments, a third guide nucleic acid of the plurality of guide nucleic acid is complementary to a third target sequence in a pX region gene. In some embodiments, a fourth guide nucleic acid of the plurality of guide nucleic acid is complementary to a fourth target sequence in a pX region gene. In some embodiments, the first target sequence, the second target sequence, the third target sequence, and the fourth target sequence are different, wherein the intervening sequences between pairs of guide nucleic acids are excised or inactivate the expression or function of the pX region gene.

In some embodiments, a first guide nucleic acid of the plurality of guide nucleic acids is complementary to a first target sequence in a HBZ or APH-2 gene. In some embodiments, a second guide nucleic acid of the plurality of guide nucleic acids is complementary to a second target sequence in a HBZ or APH-2 gene. In some embodiments, a third guide nucleic acid of the plurality of guide nucleic acid is complementary to a third target sequence in a HBZ or APH-2 gene. In some embodiments, a fourth guide nucleic acid of the plurality of guide nucleic acid is complementary to a fourth target sequence in a HBZ or APH-2 gene. In some embodiments, the first target sequence, the second target sequence, the third target sequence, and the fourth target sequence are different, wherein the intervening sequences between pairs of guide nucleic acids are excised or inactivate the expression or function of the HBZ or APH-2 gene.

In some embodiments, a first guide nucleic acid of the plurality of guide nucleic acids is complementary to a first target sequence in a Tax-1 gene. In some embodiments, a second guide nucleic acid of the plurality of guide nucleic acids is complementary to a second target sequence in a Tax-1 gene in some embodiments, a third guide nucleic acid of the plurality of guide nucleic acid is complementary to a third target sequence in a Tax 1 gene. In some embodiments, a fourth guide nucleic acid of the plurality of guide nucleic acid is complementary to a fourth target sequence in a Tax-1 gene. In some embodiments, the first target sequence, the second target sequence, the third target sequence, and the fourth target sequence are different, wherein the intervening sequences between pairs of guide nucleic acids are excised or inactivate the expression or function of the Tax-1 gene.

In some embodiments, a first guide nucleic acid of the plurality of guide nucleic acids is complementary to a first target sequence in a Tax-2 gene. In some embodiments, a second guide nucleic acid of the plurality of guide nucleic acids is complementary to a second target sequence in a Tax-2 gene. In some embodiments, a third guide nucleic acid of the plurality of guide nucleic acid is complementary to a third target sequence in a Tax-2 gene. In some embodiments, a fourth guide nucleic acid of the plurality of guide nucleic acid is complementary to a fourth target sequence in a Tax-2 gene. In some embodiments, the first target sequence, the second target sequence, the third target sequence, and the fourth target sequence are different, wherein the intervening sequences between pairs of guide nucleic acids are excised or inactivate the expression or function of the Tax-2 gene.

In some embodiments, compositions and methods described herein comprise 2, 3, 4, 5, 6, or more than 6 different gRNAs that target (e.g., hybridize or anneal to) or are complementary to a region within target nucleic acid sequences comprising. LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof.

In some embodiments, compositions and methods described herein comprise 2, 3, 4, 5, 6, or more than 6 different gRNAs that target the LTR gene. In some embodiments, compositions and methods described herein comprise 2, 3, 4, 5, 6, or more than 6 different gRNAs that target the Env gene. In some embodiments, compositions and methods described herein comprise 2, 3, 4, 5, 6, or more than 6 different gRNAs that target the Gag gene. In some embodiments, compositions and methods described herein comprise 2, 3, 4, 5, 6, or more than 6 different gRNAs that target the Pol gene. In some embodiments, compositions and methods described herein comprise 2, 3, 4, 5, 6, or more than 6 different gRNAs that target the Pro gene. In some embodiments, compositions and methods described herein comprise 2, 3, 4, 5, 6, or more than 6 different gRNAs that target the pX region gene. In some embodiments, compositions and methods described herein comprise 2, 3, 4, 5, 6, or more than 6 different gRNAs that target the HBZ gene. In some embodiments, compositions and methods described herein comprise 2, 3, 4, 5, 6, or more than 6 different gRNAs that target the APH-2 gene. In some embodiments, compositions and methods described herein comprise 2, 3, 4, 5, 6, or more than 6 different gRNAs that target the Tax-1 gene. In some embodiments, compositions and methods described herein comprise 2, 3, 4, 5, 6, or more than 6 different gRNAs that target the Tax-2 gene.

In some embodiments, compositions and methods described herein comprise 1, 2, 3, 4, 5, 6, or more than 6 different gRNAs that target the 5′-LTR gene and 1, 2, 3, 4, 5, 6, or more than 6 different gRNAs that target the 3′-LTR gene. In some embodiments, compositions and methods described herein comprise 1, 2, 3, 4, 5, 6, or more than 6 different gRNAs that target the 5′-LTR or 3′-LTR gene and 1, 2, 3, 4, 5, 6, or more than 6 different gRNAs that target the Gag gene. In some embodiments, compositions and methods described herein comprise 1, 2, 3, 4, 5, 6, or more than 6 different gRNAs that target the 5′-LTR or 3′-LTR gene and 1, 2, 3, 4, 5, 6, or more than 6 different gRNAs that target the Pol gene. In some embodiments, compositions and methods described herein comprise 1, 2, 3, 4, 5, 6, or more than 6 different gRNAs that target the 5′-LTR or 3′-LTR gene and 1, 2, 3, 4, 5, 6, or more than 6 different gRNAs that target the Pro gene. In some embodiments, compositions and methods described herein comprise 1, 2, 3, 4, 5, 6, or more than 6 different gRNAs that target the 5′-LTR or 3′-LTR gene and 1, 2, 3, 4, 5, 6, or more than 6 different gRNAs that target the Env gene. In some embodiments, compositions and methods described herein comprise 1, 2, 3, 4, 5, 6, or more than 6 different gRNAs that target the 5′-LTR or 3′-LTR gene and 1, 2, 3, 4, 5, 6, or more than 6 different gRNAs that target the pX region gene. In some embodiments, compositions and methods described herein comprise 1, 2, 3, 4, 5, 6, or more than 6 different gRNAs that target the 5′-LTR or 3′-LTR gene and 1, 2, 3, 4, 5, 6, or more than 6 different gRNAs that target the HBZ gene. In some embodiments, compositions and methods described herein comprise 1, 2, 3, 4, 5, 6, or more than 6 different gRNAs that target the 5′-LTR or 3′-LTR gene and 1, 2, 3, 4, 5, 6, or more than 6 different gRNAs that target the APH-2 gene. In some embodiments, compositions and methods described herein comprise 1, 2, 3, 4, 5, 6, or more than 6 different gRNAs that target the 5′-LTR or 3′-LTR gene and 1, 2, 3, 4, 5, 6, or more than 6 different gRNAs that tartlet the Tax-1 gene. In some embodiments, compositions and methods described herein comprise 1, 2, 3, 4, 5, 6, or more than 6 different gRNAs that target the 5′-LTR or 3′-LTR gene and 1, 2, 3, 4, 5, 6, or more than 6 different gRNAs that target the Tax-2 gene.

In some embodiments, compositions and methods described herein comprise 1, 2, 3, 4, 5, 6, or more than 6 different gRNAs that target the Gag gene and 1, 2, 3, 4, 5, 6, or more than 6 different gRNAs that target the Pol gene. In some embodiments, compositions and methods described herein comprise 1, 2, 3, 4, 5, 6, or more than 6 different gRNAs that target the Gag gene and 1, 2, 3, 4, 5, 6, or more than F, different gRNAs that target the Pro gene. In some embodiments, compositions and methods described herein comprise 1, 2, 3, 4, 5, 6, or more than 6 different gRNAs that target the Gag gene and 1, 2, 3, 4, 5, 6, or more than 6 different gRNAs that target the Env gene. In some embodiments, compositions and methods described herein comprise 1, 2, 3, 4, 5, 6, or more than 6 different gRNAs that target the Gag gene and 1, 2, 3, 4, 5, 6, or more than 6 different gRNAs that target the pX region gene. In some embodiments, compositions and methods described herein comprise 1, 2, 3, 4, 5, 6, or more than 6 different gRNAs that target the Gag gene and 1, 2, 3, 4, 5, 6, or more than 6 different gRNAs that target the HBZ gene. In some embodiments, compositions and methods described herein comprise 1, 2, 3, 4, 5, 6, or more than 6 different gRNAs that target the Gag gene and 1, 2, 3, 4, 5, 6, or more than 6 different gRNAs that target the APH-2 gene. In some embodiments, compositions and methods described herein comprise 1, 2, 3, 4, 5, 6, or more than 6 different gRNAs that target the Gag gene and 1, 2, 3, 4, 5, 6, or more than 6 different gRNAs that target the Tax-1 gene. In some embodiments, compositions and methods described herein comprise 1, 2, 3, 4, 5, 6, or more than 6 different gRNAs that target the Gag gene and 1, 2, 3, 4, 5, 6, or more than 6 different gRNAs that target the Tax-2 gene.

In some embodiments, compositions and methods described herein comprise 1, 2, 3, 4, 5, 6, or more than 6 different gRNAs that target the Pol gene and 1, 2, 3, 4, 5, 6, or more than 6 different gRNAs that target the Pro gene. In some embodiments, compositions and methods described herein comprise 1, 2, 3, 4, 5, 6, or more than 6 different gRNAs that target the Pol gene and 1, 2, 3, 4, 5, 6, or more than 6 different gRNAs that target the Env gene. In some embodiments, compositions and methods described herein comprise 1, 2, 3, 4, 5, 6, or more than 6 different gRNAs that target the Pol gene and 1, 2, 3, 4, 5, 6, or more than 6, different gRNAs that target the pX region gene. In some embodiments, compositions and methods described herein comprise 1, 2, 3, 4, 5, 6, or more than 6 different gRNAs that target the Pol gene and 1, 2, 3, 4, 5, 6 or more than 6 different gRNAs that target the HBZ gene. In some embodiments, compositions and methods described herein comprise 1, 2, 3, 4, 5, 6, or more than 6 different gRNAs that target the Pol gene and 1, 2, 3, 4, 5, 6, or more than 6 different gRNAs that target the APH-2 gene. In some embodiments, compositions and methods described herein comprise 1, 2, 3, 4, 5, 6, or more than 6 different gRNAs that target the Pol gene and 1, 2, 3, 4, 5, 6, or more than 6 different gRNAs that target the Tax-1 gene. In some embodiments, compositions and methods described herein comprise 1, 2, 3, 4, 5, 6, or more than 6 different gRNAs that target the Pol gene and 1, 2, 3, 4, 5, 6, or more than 6 different gRNAs that target the Tax-2 gene.

In some embodiments, compositions and methods described herein comprise 1, 2, 3, 4, 5, 6, or more than 6 different gRNAs that target the Pro gene and 1, 2, 3, 4, 5, 6, or more than 6 different gRNAs that target the Env gene. In some embodiments, compositions and methods described herein comprise 1, 2, 3, 4, 5, 6, or more than 6 different gRNAs that target the Pro gene and 1, 2, 3, 4, 5, 6, or more than 6 different gRNAs that target the pX region gene. In some embodiments, compositions and methods described herein comprise 1, 2, 3, 4, 5, 6, or more than 6 different gRNAs that target the Pro gene and 1, 2, 3, 4, 5, 6, or more than 6 different gRNAs that target the HBZ gene. In some embodiments, compositions and methods described herein comprise 1, 2, 3, 4, 5, 6, or more than 6 different gRNAs that target the Pro gene and 1, 2, 3, 4, 5, 6, or more than 6 different gRNAs that target the APH-2 gene. In some embodiments, compositions and methods described herein comprise 1, 2, 3, 4, 5, 6, or more than 6 different gRNAs that target the Pro gene and 1, 2, 3, 4, 5, 6, or more than 6 different gRNAs that target the Tax-1 gene. In some embodiments, compositions and methods described herein comprise 1, 2, 3, 4, 5, 6, or more than 6 different gRNAs that target the Pro gene and 1, 2, 3, 4, 5, 6, or more than 6 different gRNAs that target the Tax-2 gene.

In some embodiments, compositions and methods described herein comprise 1, 2, 3, 4, 5, 6, or more than 6 different gRNAs that target the Env gene and 1, 2, 3, 4, 5, 6, or more than 6 different gRNAs that target the pX region gene. In some embodiments, compositions and methods described herein comprise 1, 2, 3, 4, 5, 6, or more than 6 different gRNAs that target the Env gene and 1, 2, 3, 4, 5, 6, or more than 6 different gRNAs that target the HBZ gene. In some embodiments, compositions and methods described herein comprise 1, 2, 3, 4, 5, 6, or more than 6 different gRNAs that target the Env gene and 1, 2, 3, 4, 5, 6, or more than 6 different gRNAs that target the APH-2 gene. In some embodiments, compositions and methods described herein comprise 1, 2, 3, 4, 5, 6, or more than 6 different gRNAs that target the Env gene and 1, 2, 3, 4, 5, 6, or more than 6 different gRNAs that target the Tax-1 gene. In some embodiments, compositions and methods described herein comprise 1, 2, 3, 4, 5, 6, or more than 6 different gRNAs that target the Env gene and 1, 2, 3, 4, 5, 6, or more than 6 different gRNAs that target the Tax-2 gene.

In some embodiments, compositions and methods described herein comprise 1, 2, 3, 4, 5, 6, or more than 6 different gRNAs that target the pX region gene and 1, 2, 3, 4, 5, 6, or more than 6 different gRNAs that target the HBZ gene. In some embodiments, compositions and methods described herein comprise 1, 2, 3, 4, 5, 6, or more than 6 different gRNAs that target the pX region gene and 1, 2, 3, 4, 5, 6, or more than 6 different gRNAs that target the APH-2 gene. In some embodiments, compositions and methods described herein comprise 1, 2, 3, 4, 5, 6, or more than 6 different gRNAs that target the pX region gene and 1, 2, 3, 4, 5, 6, or more than 6 different gRNAs that target the Tax-1 gene. In some embodiments, compositions and methods described herein comprise 1, 2, 3, 4, 5, 6, or more than 6 different gRNAs that target the pX region gene and 1, 2, 3, 4, 5, 6, or more than 6 different gRNAs that target the Tax-2 gene.

In some embodiments, compositions and methods described herein comprise 1, 2, 3, 4, 5, 6, or more than 6 different gRNAs that target the HBZ or APH-2 gene and 1, 2, 3, 4, 5, 6, or more than 6 different gRNAs that target the Tax-1 gene. In some embodiments, compositions and methods described herein comprise 1, 2, 3, 4, 5, 6, or more than 6 different gRNAs that target the HBZ or APN-2 gene and 1, 2, 3, 4, 5, 6, or more than 6 different gRNAs that target the Tax-2 gene.

In some embodiments, compositions and methods described herein comprise 1, 2, 3, 4, 5, 6, or more than 6 different gRNAs that target the 5′-LTR gene and 1, 2, 3, 4, 5, 6, or more than 6 different gRNAs that target the 3′-LTR gene and 1, 2, 3, 4, 5, 6, or more than 6 different gRNAs that target the Gag gene and 1, 2, 3, 4, 5, 6, or more than 6 different gRNAs that target the Pol gene and 1, 2, 3, 4, 5, 6, or more than 6 different gRNAs that target the Env gene and 1, 2, 3, 4, 5, 6, or more than 6 different gRNAs that target the pX region gene or more than 6 different gRNAs that target the HBZ or APH-2 gene or more than 6 different gRNAs that target the Tax-1 gene or more than 6 different gRNAs that target the Tax-2 gene.

In some embodiments, compositions and methods described herein comprise 2 different gRNAs that target the 5′-LTR gene and 1 gRNA that targets the 3′-LTR gene. In some embodiments, compositions and methods described herein comprise 2 different gRNAs that target the 5′-LTR gene and 2 different gRNAs that target the 3′-LTR gene. In some embodiments, compositions and methods described herein comprise 1 gRNA that targets the 5′-LTR gene and 2 different gRNAs that target the 3′-LTR gene. In some embodiments, compositions and methods described herein comprise 2 different gRNAs that target the 5′- or 3′-LTR gene and 1 gRNA that targets the Gag gene. In some embodiments, compositions and methods described herein comprise 2 different gRNAs that target the 5′- or 3′-LTR gene and 2 different gRNAs that target the Gag gene. In some embodiments, compositions and methods described herein comprise 1 gRNA that targets the 5′- or 3′-LTR gene and 2 different gRNAs that targets the Gag gene. In some embodiments, compositions and methods described herein comprise 2 different gRNAs that target the 5′- or 3′-LTR gene and 1 gRNA that targets the Pol gene. In some embodiments, compositions and methods described herein comprise 2 different gRNAs that target the 5′- or 3′-LTR gene and 2 different gRNAs that target the Pol gene. In some embodiments, compositions and methods described herein comprise 1 gRNA that targets the 5′- or 3′-LTR gene and 2 different gRNAs that targets the Pol gene. In some embodiments, compositions and methods described herein comprise 2 different gRNAs that target the 5′- or 3′-LTR gene and 1 gRNA that targets the Env gene. In some embodiments, compositions and methods described herein comprise 2 different gRNAs that target the 5′- or 3′-LTR gene and 2 different gRNAs that target the Env gene. In some embodiments, compositions and methods described herein comprise 1 gRNA that targets the 5′- or 3′-LTR gene and 2 different gRNAs that targets the Env gene. In some embodiments, compositions and methods described herein comprise 2 different gRNAs that target the 5′- or 3′-LTR gene and 1 gRNA that targets the pX region gene. In some embodiments, compositions and methods described herein comprise 2 different gRNAs that target the 5′- or 3′-LTR gene and 2 different gRNAs that target the pX region gene. In some embodiments, compositions and methods described herein comprise 1 gRNA that targets the 5′- or 3′-LTR gene and 2 different gRNAs that targets the pX region gene. In some embodiments, compositions and methods described herein comprise 2 different gRNAs that target the 5 or 3′-LTR gene and 1 gRNA that targets the HBZ or APH-2 gene. In some embodiments, compositions and methods described herein comprise 2 different gRNAs that target the 5′- or 3′-LTR gene and 2 different gRNAs that target the HBZ or APH-2 gene. In some embodiments, compositions and methods described herein comprise 1 gRNA that targets the 5′- or 3′-LTR gene and 2 different gRNAs that targets the HBZ or APH-2 gene to some embodiments, compositions and methods described herein comprise 2 different gRNAs that target the 5′- or 3′-LTR gene and 1 gRNA that targets the Tax-1 gene. In some embodiments, compositions and methods described herein comprise 2 different gRNAs that target the 5′- or 3′-LTR gene and 2 different gRNAs that target the Tax-1 gene. In some embodiments, compositions and methods described herein comprise 1 gRNA that targets the 5′- or 3′-LTR gene and 2 different gRNAs that targets the Tax-1 gene. In some embodiments, compositions and methods described herein comprise 2 different gRNAs that target the 5′- or 3′-LTR gene and 1 gRNA that targets the Tax-2 gene. In some embodiments, compositions and methods described herein comprise 2 different gRNAs that target the 5′- or 3′-LTR gene and 2 different gRNAs that target the Tax-2 gene. In some embodiments, compositions and methods described herein comprise 1 gRNA that targets the 5′- or 3′-LTR gene and 2 different gRNAs that targets the Tax-2 gene.

In some embodiments, compositions and methods described herein comprise 2 different gRNAs that target the Gag gene and 1 gRNA that targets the Pol gene. In some embodiments, compositions and methods described herein comprise 2 different gRNAs that target the Gag gene and 2 different gRNAs that target the Pol gene. In some embodiments, compositions and methods described herein comprise 1 gRNA that targets the Gag gene and 2 different gRNAs that targets the Pol gene. In some embodiments, compositions and methods described herein comprise 2 different gRNAs that target the Gag gene and 1 gRNA that targets the Pro gene. In some embodiments, compositions and methods described herein comprise 2 different gRNAs that target the Gag gene and 2 different gRNAs that target the Pro gene. In some embodiments, compositions and methods described herein comprise 1 gRNA that targets the Gag gene and 2 different gRNAs that targets the Pro gene. In some embodiments, compositions and methods described herein comprise 2 different gRNAs that target the Gag gene and 1 gRNA that targets the Env gene. In some embodiments, compositions and methods described herein comprise 2 different gRNAs that target the Gag gene and 2 different gRNAs that target the Env gene. In some embodiments, compositions and methods described herein comprise 1 gRNA that targets the Gag gene and 2 different gRNAs that targets the Env gene. In some embodiments, compositions and methods described herein comprise 2 different gRNAs that target the Gag gene and 1 gRNA that targets the pX region gene. In some embodiments, compositions and methods described herein comprise 2 different gRNAs that target the Gag gene and 2 different gRNAs that target the pX region gene. In some embodiments, compositions and methods described herein comprise 1 gRNA that targets the Gag gene and 2 different gRNAs that targets the pX region gene. In some embodiments, compositions and methods described herein comprise 2 different gRNAs that target the Gag gene and 1 gRNA that targets the HBZ or APH-2 gene. In some embodiments, compositions and methods described herein comprise 2 different gRNAs that target the Gag gene and 2 different gRNAs that target the HBZ or APH-2 gene. In some embodiments, compositions and methods described herein comprise 1 gRNA that targets the Gag gene and 2 different gRNAs that targets the HBZ or APH-2 gene. In some embodiments, compositions and methods described herein comprise 2 different gRNAs that target the Gag gene and 1 gRNA that targets the Tax-1 gene. In some embodiments, compositions and methods described herein comprise 2 different gRNAs that target the Gag gene and 2 different gRNAs that target the Tax-1 gene. In some embodiments, compositions and methods described herein comprise 1 gRNA that targets the Gag gene and 2 different gRNAs that targets the Tax-1 gene. In some embodiments, compositions and methods described herein comprise 2 different gRNAs that target the Gag gene and 1 gRNA that targets the Tax-2 gene. In some embodiments, compositions and methods described herein comprise 2 different gRNAs that target the Gag gene and 2 different gRNAs that target the Tax-2 gene. In some embodiments, compositions and methods described herein comprise 1 gRNA that targets the Gag gene and 2 different gRNAs that targets the Tax-2 gene.

In some embodiments, compositions and methods described herein comprise 2 different gRNAs that target the Pol gene and 1 gRNA that targets the Pro gene. In some embodiments, compositions and methods described herein comprise 2 different gRNAs that target the Pol gene and 2 different gRNAs that target the Pro gene. In some embodiments, compositions and methods described herein comprise 1 gRNA that targets the Pol gene and 2 different gRNAs that targets the Pro gene. In some embodiments, compositions and methods described herein comprise 2 different gRNAs that target the Pol gene and 1 gRNA that targets the Env gene. In some embodiments, compositions and methods described herein comprise 2 different gRNAs that target the Pol gene and 2 different gRNAs that target the Env gene. In some embodiments, compositions and methods described herein comprise 1 gRNA that targets the Pol gene and 2 different gRNAs that targets the Env gene. In some embodiments, compositions and methods described herein comprise 2 different gRNAs that target the Pol gene and 1 gRNA that targets the pX region gene. In some embodiments, compositions and methods described herein comprise 2 different gRNAs that target the Pol gene and 2 different gRNAs that target the pX region gene. In some embodiments, compositions and methods described herein comprise 1 gRNA that targets the Pol gene and 2 different gRNAs that targets the pX region gene. In some embodiments, compositions and methods described herein comprise 2 different gRNAs that target the Pol gene and 1 gRNA that targets the HBZ or APH-2 gene. In some embodiments, compositions and methods described herein comprise 2 different gRNAs that target the Pol gene and 2 different gRNAs that target the HBZ or APH-2 gene. In some embodiments, compositions and methods described herein comprise 1 gRNA that targets the Pol gene and 2 different gRNAs that targets the HBZ or APH-2 gene. In some embodiments, compositions and methods described herein comprise 2 different gRNAs that target the Pol gene and 1 gRNA that targets the Tax-1 gene. In some embodiments, compositions and methods described herein comprise 2 different gRNAs that target the Pol gene and 2 different gRNAs that target the Tax-1 gene. In some embodiments, compositions and methods described herein comprise 1 gRNA that targets the Pol gene and 2 different gRNAs that targets the Tax-1 gene. In some embodiments, compositions and methods described herein comprise 2 different gRNAs that target the Pol gene and 1 gRNA that targets the Tax-2 gene. In some embodiments, compositions and methods described herein comprise 2 different gRNAs that target the Pol gene and 2 different gRNAs that target the Tax-2 gene. In some embodiments, compositions and methods described herein comprise 1 gRNA that targets the Pol gene and 2 different gRNAs that targets the Tax-2 gene.

In some embodiments, compositions and methods described herein comprise 2 different gRNAs that target the Pro gene and 1 gRNA that targets the Env gene. In some embodiments, compositions and methods described herein comprise 2 different gRNAs that target the Pro gene and 2 different gRNAs that target the Env gene. In some embodiments, compositions and methods described herein comprise 1 gRNA that targets the Pro gene and 2 different gRNAs that targets the Env gene. In some embodiments, compositions and methods described herein comprise 2 different gRNAs that target the Pro gene and 1 gRNA that targets the pX region gene. In some embodiments, compositions and methods described herein comprise 2 different gRNAs that target the Pro gene and 2 different gRNAs that target the pX region gene. In some embodiments, compositions and methods described herein comprise 1 gRNA that targets the Pro gene and 2 different gRNAs that targets the pX region gene. In some embodiments, compositions and methods described herein comprise 2 different gRNAs that target the Pro gene and 1 gRNA that targets the HBZ or APH-2 gene. In some embodiments, compositions and methods described herein comprise 2 different gRNAs that target the Pro gene and 2 different gRNAs that target the HBZ or APH-2 gene. In some embodiments, compositions and methods described herein comprise 1 gRNA that targets the Pro gene and 2 different gRNAs that targets the HBZ or APH-2 gene. In some embodiments, compositions and methods described herein comprise 2 different gRNAs that target the Pro gene and 1 gRNA that targets the Tax-1 gene. In some embodiments, compositions and methods described herein comprise 2 different gRNAs that target the Pro gene and 2 different gRNAs that target the Tax-1 gene. In some embodiments, compositions and methods described harem comprise 1 gRNA that targets the Pro gene and 2 different gRNAs that targets the Tax-1 gene. In some embodiments, compositions and methods described herein comprise 2 different gRNAs that target the Pro gene and 1 gRNA that targets the Tax-2 gene. In some embodiments, compositions and methods described herein comprise 2 different gRNAs that target the Pro gene and 2 different gRNAs that target the Tax-2 gene. In some embodiments, compositions and methods described herein comprise 1 gRNA that targets the Pro gene and 2 different gRNAs that targets the Tax-2 gene.

In some embodiments, compositions and methods described herein comprise 2 different gRNAs that target the Env gene and 1 gRNA that targets the pX region gene. In some embodiments, compositions and methods described herein comprise 2 different gRNAs that target the Env gene and 2 different gRNAs that target the pX region gene. In some embodiments, compositions and methods described herein comprise 1 gRNA that targets the Env gene and 2 different gRNAs that targets the pX region gene. In some embodiments, compositions and methods described herein comprise 2 different gRNAs that target the Env gene and 1 gRNA that targets the HIV or APH-2 gene. In some embodiments, compositions and methods described herein comprise 2 different gRNAs that target the Env gene and 2 different gRNAs that target the HBZ or APH-2 gene. In some embodiments, compositions and methods described herein comprise 1 gRNA that targets the Env gene and 2 different gRNAs that targets the HBZ or APH-2 gene. In some embodiments, compositions and methods described herein comprise 2 different gRNAs that target the Env gene and 1 gRNA that targets the Tax-1 gene. In some embodiments, compositions and methods described herein comprise 2 different gRNAs that target the Env gene and 2 different gRNAs that target the Tax-1 gene. In some embodiments, compositions and methods described herein comprise 1 gRNA that targets the Env gene and 2 different gRNAs that targets the Tax-1 gene. In some embodiments, compositions and methods described herein comprise 2 different gRNAs that target the Env gene and 1 gRNA that targets the Tax-2 gene. In some embodiments, compositions and methods described herein comprise 2 different gRNAs that target the Env gene and 2 different gRNAs that target the Tax-2 gene. In some embodiments, compositions and methods described herein comprise 1 gRNA that targets the Env gene and 2 different gRNAs that targets the Tax-2 gene.

In some embodiments, compositions and methods described herein comprise 2 different gRNAs that target the pX region gene and 1 gRNA that targets the HBZ, or APH-2 gene. In some embodiments, compositions and methods described herein comprise 2 different gRNAs that target the pX region gene and 2 different gRNAs that target the HBZ or APH-2 gene. In some embodiments, compositions and methods described herein comprise 1 gRNA that targets the pX region gene and 2 different gRNAs that targets the HBZ or APH-2 gene. In some embodiments, compositions and methods described herein comprise 2 different gRNAs that target the pX region gene and 1 gRNA that targets the Tax-1 gene. In some embodiments, compositions and methods described herein comprise 2 different gRNAs that target the pX region gene and 2 different gRNAs that target the Tax-1 gene. In some embodiments, compositions and methods described herein comprise 1 gRNA that targets the pX region gene and 2 different gRNAs that targets the Tax-1 gene. In some embodiments, compositions and methods described herein comprise 2 different gRNAs that target the pX region gene and 1 gRNA that targets the Tax-2 gene. In some embodiments, compositions and methods described herein comprise 2 different gRNAs that target the pX region gene and 2 different gRNAs that target the Tax-2 gene. In some embodiments, compositions and methods described herein comprise 1 gRNA that targets the pX region gene and 2 different gRNAs that targets the Tax-2 gene.

In some embodiments, compositions and methods described herein comprise 2 different gRNAs that target the HBZ or APH-2 gene and 1 gRNA that targets the Tax-1 gene. In some embodiments, compositions and methods described herein comprise 2 different gRNAs that target the HBZ or APH-2 gene and 2 different gRNAs that target the Tax-1 gene. In some embodiments, compositions and methods described herein comprise 1 gRNA that targets the HBZ or APH-2 gene and 2 different gRNAs that targets the Tax-1 gene. In some embodiments, compositions and methods described herein comprise 2 different gRNAs that target the HBZ or APH-2 gene and 1 gRNA that targets the Tax-2 gene. In some embodiments, compositions and methods described herein comprise 2 different gRNAs that target the HBZ or APH-2 gene and 2 different gRNAs that target the Tax-2 gene. In some embodiments, compositions and methods described herein comprise 1 gRNA that targets the HBZ or APH-2 gene and 2 different gRNAs that targets the Tax-2 gene.

In some embodiments, compositions and methods described herein comprise 2 different gRNAs that target the Tax-1 gene and 1 gRNA that targets the Tax-2 gene. In some embodiments, compositions and methods described herein comprise 2 different gRNAs that target the Tax-1 the and 2 different gRNAs that target the Tae-2 gene. In some embodiments, compositions and methods described herein comprise 1 gRNA that targets the Tax-1 gene and 2 different gRNAs that targets the Tax-2 gene.

In some embodiments, compositions and methods described herein comprise 1, 2, 3, 4, 5, 6 or more than 6 different gRNAs that hybridize to the 5′-LTR gene and 1, 2, 3, 4, 5, 6, or more than 6 different gRNAs that hybridize to the 3′-LTR gene. In some embodiments, compositions and methods described herein comprise 1, 2, 3, 4, 5, 6, or more than 6 different gRNAs that hybridize to the 5′- or 3′-LTR gene and 1, 2, 3, 4, 5, 6, or more than 6 different gRNAs that hybridize to the Gag gene. In some embodiments, compositions and methods described herein comprise 1, 2, 3, 4, 5, 6, or more than 6 different gRNAs that hybridize to the 5′- or 3′-LTR gene and 1, 2, 3, 4, 5, 6, or more than 6 different gRNAs that hybridize to the Pol gene. In some embodiments, compositions and methods described herein comprise 1, 2, 3, 4, 5, 6, or more than 6 different gRNAs that hybridize to the 5′- or 3′-LTR gene and 1, 2, 3, 4, 5, 6, or more than 6 different gRNAs that hybridize to the Pro gene. In some embodiments, compositions and methods described herein comprise 1, 2, 3, 4, 5, 6, or more than 6 different gRNAs that hybridize to the 5′- or 3′-LTR gene and 1, 2, 3, 4, 5, 6, or more than 6 different gRNAs that hybridize to the Env gene. In some embodiments, compositions and methods described herein comprise 1, 2, 3, 4, 5, 6, or more than 6 different gRNAs that hybridize to the 5′- or 3′-LTR gene and 1, 2, 3, 4, 5, 6, or more than 6 different gRNAs that hybridize to the pX region gene. In some embodiments, compositions and methods described herein comprise 1, 2, 3, 4, 5, 6, or more than 6 different gRNAs that hybridize to the 5′- or 3′-LTR gene and 1, 2, 3, 4, 5, 6, or more than 6 different gRNAs that hybridize to the HBZ or APH-2 gene. In some embodiments, compositions and methods described herein comprise 1, 2, 3, 4, 5, 6, or more than 6 different gRNAs that hybridize to the 5′- or 3′-LTR gene and 1, 2, 3, 4, 5, 6, or more than 6 different gRNAs that hybridize to the Tax-1 gene. In some embodiments, compositions and methods described herein comprise 1, 2, 3, 4, 5, 6, or more than 6 different gRNAs that hybridize to the 5′- or 3′-LTR gene and 1, 2, 3, 4, 5, 6, or more than 6 different gRNAs that hybridize to the Tax-2 gene.

In some embodiments, compositions and methods described herein comprise 1, 2, 3, 4, 5, 6, or more than 6 different gRNAs that hybridize to the Gag gene and 1, 2, 3, 4, 5, 6, or more than 6 different gRNAs that hybridize to the Pol gene. In some embodiments, compositions and methods described herein comprise 1, 2, 3, 4, 5, 6, or more than 6 different gRNAs that hybridize to the Gag gene and 1, 2, 3, 4, 5, 6, or more than 6 different gRNAs that hybridize to the Pro gene. In some embodiments, compositions and methods described herein comprise 1, 2, 3, 4, 5, 6, or more than 6 different gRNAs that hybridize to the Gag gene and 1, 2, 3, 4, 5, 6, or more than 6 different gRNAs that hybridize to the Env gene. In some embodiments, compositions and methods described herein comprise 1, 2, 3, 4, 5, 6, or more than 6 different gRNAs that hybridize to the Gag gene and 1, 2, 3, 4, 5, 6, or more than 6 different gRNAs that hybridize to the pX region gene. In some embodiments, compositions and methods described herein comprise 1, 2, 3, 4, 5, 6, or more than 6 different gRNAs that hybridize to the Gag gene and 1, 2, 3, 4, 5, 6, or more than 6 different gRNAs that hybridize to the HBZ or APH-2 gene. In some embodiments, compositions and methods described herein comprise 1, 2, 3, 4, 5, 6, or more than 6 different gRNAs that hybridize to the Gag gene and 1, 2, 3, 4, 5, 6, or more than 6 different gRNAs that hybridize to the Tax-1 gene. In some embodiments, compositions and methods described herein comprise 1, 2, 3, 4, 5, 6, or more than 6 different gRNAs that hybridize to the Gag gene and 1, 2, 3, 4, 5, 6, or more than 6 different gRNAs that hybridize to the Tax-2 gene.

In some embodiments, compositions and methods described herein comprise 1, 2, 3, 4, 5, 6, or more than 6 different gRNAs that hybridize to the Vol gene and 1, 2, 3, 4, 5, 6, or more than 6 different gRNAs that hybridize to the Pro gene. In some embodiments, compositions and methods described herein comprise 1, 2, 3, 4, 5, 6, or more than 6 different gRNAs that hybridize to the Pol gene and 1, 2, 3, 4, 5, 6, or more than 6 different gRNAs that hybridize to the Env gene. In some embodiments, compositions and methods described herein comprise 1, 2, 3, 4, 5, 6, or more than 6 different gRNAs that hybridize to the Pol gene and 1, 2, 3, 4, 5, 6, or more than 6 different gRNAs that hybridize to the pX region gene. In some embodiments, compositions and methods described herein comprise 1, 2, 3, 4, 5, 6, or more than 6 different gRNAs that hybridize to the Pol gene and 1, 2, 3, 4, 5, 6, or more than 6 different gRNAs that hybridize to the HBZ or APH-2 gene. In some embodiments, compositions and methods described herein comprise 1, 2, 3, 4, 5, 6, or more than 6 different gRNAs that hybridize to the Pol gene and 1, 2, 3, 4, 5, 6, or more than 6 different gRNAs that hybridize to the Tax-1 gene. In some embodiments, compositions and methods described herein comprise 1, 2, 3, 4, 5, 6, or more than 6 different gRNAs that hybridize to the Pol gene and 1, 2, 3, 4, 5, 6, or more than 6 different gRNAs that hybridize to the Tax-2 gene.

In some embodiments, compositions and methods described herein comprise 1, 2, 3, 4, 5, 6, or more than 6 different gRNAs that hybridize to the Pro gene and 1, 2, 3, 4, 5, 6, or more than 6 different gRNAs that hybridize to the Env gene. In some embodiments, compositions and methods described herein comprise 1, 2, 3, 4, 5, 6, or more than 6 different gRNAs that hybridize to the Pro gene and 1, 2, 3, 4, 5, 6, or more than 6 different gRNAs that hybridize to the pX region gene. In some embodiments, compositions and methods described herein comprise 1, 2, 3, 4, 5, 6, or more than 6 different gRNAs that hybridize to the Pro gene and 1, 2, 3, 4, 5, 6, or more than 6 different gRNAs that hybridize to the HBZ or APH-2 gene. In some embodiments, compositions and methods described herein comprise 1, 2, 3, 4, 5, 6, or more than 6 different gRNAs that hybridize to the Pro gene and 1, 2, 3, 4, 5, 6, or more than 6 different gRNAs that hybridize to the Tax-1 gene in some embodiments, compositions and methods described herein comprise 1, 2, 3, 4, 5, 6, or more than 6 different gRNAs that hybridize to the Pro gene and 1, 2, 3, 4, 5, 6, or more than 6 different gRNAs that hybridize to the Tax-2 gene.

In some embodiments, compositions and methods described herein comprise 1, 2, 3, 4, 5, 6, or more than 6 different gRNAs that hybridize to the Env gene and 1, 2, 3, 4, 5, 6, or more than 6 different gRNAs that hybridize to the pX region gene. In some embodiments, compositions and methods described herein comprise 1, 2, 3, 4, 5, 6, or more than 6 different gRNAs that hybridize to the Env gene and 1, 2, 3, 4, 5, 6, or more than 6 different gRNAs that hybridize to the HBZ or APH-2 gene. In some embodiments, compositions and methods described herein comprise 1, 2, 3, 4, 5, 6, or more than 6 different gRNAs that hybridize to the Env gene and 1, 2, 3, 4, 5, 6, or more than 6 different gRNAs that hybridize to the Tax-1 gene. In some embodiments, compositions and methods described herein comprise 1, 2, 3, 4, 5, 6, or more than 6 different gRNAs that hybridize to the Env gene and 1, 2, 3, 4, 5, 6, or more than 6 different gRNAs that hybridize to the Tax-2 gene.

In some embodiments, compositions and methods described herein comprise 1, 2, 3, 4, 5, 6, or more than 6 different gRNAs that hybridize to the pX region gene and 1, 2, 3, 4, 5, 6, or more than 6 different gRNAs that hybridize to the HBZ or APH-2 gene. In some embodiments, compositions and methods described herein comprise 1, 2, 3, 4, 5, 6, or more than 6 different gRNAs that hybridize to the pX region gene and 1, 2, 3, 4, 5, 6, or more than 6 different gRNAs that hybridize to the Tax-1 gene. In some embodiments, compositions and methods described herein comprise 1, 2, 3, 4, 5, 6, or more than 6 different gRNAs that hybridize to the pX region gene and 1, 2, 3, 4, 5, 6, or more than 6 different gRNAs that hybridize to the Tax-2 gene.

In some embodiments, compositions and methods described herein comprise 1, 2, 3, 4, 5, 6, or more than 6 different gRNAs that hybridize to the HBZ or APH-2 gene and 1, 2, 3, 4, 5, 6, or more than 6 different gRNAs that hybridize to the Tax-1 gene. In some embodiments, compositions and methods described herein comprise 1, 2, 3, 4, 5, 6, or more than 6 different gRNAs that hybridize to the HBZ or APH-2 gene and 1, 2, 3, 4, 5, 6, or more than 6 different gRNAs that hybridize to the Tax-2 gene.

In some embodiments, compositions and methods described herein comprise 1, 2, 3, 4, 5, 6, or more than 6 different gRNAs that hybridize to the Tax-1 gene and 1, 2, 3, 4, 5, 6, or more than 6 different gRNAs that hybridize to the Tax-2 gene.

Provided herein, in certain embodiments, are methods and compositions for targeting the LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof, using at least one guide nucleic acid or a plurality of guide nucleic acids.

In some embodiments, a first guide nucleic acid of the plurality of guide nucleic acids is complementary to a first target sequence in a 5′- and/or 3′-LTR gene in some embodiments, a second guide nucleic acid of the plurality of guide nucleic acids is complementary to a second target sequence in a 5′- and/or 3′-LTR gene. In some embodiments, a third guide nucleic acid of the plurality of guide nucleic acid is complementary to a third target sequence in a 5′- and/or 3′-LTR gene. In some embodiments, a fourth guide nucleic acid of the plurality of guide nucleic acid is complementary to a fourth target sequence in a 5′- and/or 3′-LTR gene. In some embodiments, the first target sequence, the second target sequence, the third target sequence, and the fourth target sequence are different, wherein the intervening sequences between pairs of guide nucleic acids are excised or inactivate the expression or function of the 5′- and/or 3′LTR gene.

In some embodiments, a first guide nucleic acid of the plurality of guide nucleic acids is complementary to a first target sequence in a Gag gene. In some embodiments, a second guide nucleic acid of the plurality of guide nucleic acids is complementary to a second target sequence in a Gag gene. In some embodiments, a third guide nucleic acid of the plurality of guide nucleic acid is complementary to a third target sequence in a Gag gene. In some embodiments, a fourth guide nucleic acid of the plurality of guide nucleic acid is complementary to a fourth target sequence in a Gag gene. In some embodiments, the first target sequence, the second target sequence, the third target sequence, and the fourth target sequence are different, wherein the intervening sequences between pairs of guide nucleic acids are excised or inactivate the expression or function of the Gag gene.

In some embodiments, a first guide nucleic acid of the plurality of guide nucleic acids is complementary to a first target sequence in a Pol gene. In some embodiments, a second guide nucleic acid of the plurality of guide nucleic acids is complementary to a second target sequence in a Pol gene. In some embodiments, a third guide nucleic acid of the plurality of guide nucleic acid is complementary to a third target sequence in a Pol gene. In some embodiments, a fourth guide nucleic acid of the plurality of guide nucleic acid is complementary to a fourth target sequence in a Pol gene. In some embodiments, the first target sequence, the second target sequence, the third target sequence, and the fourth target sequence are different, wherein the intervening sequences between pairs of guide nucleic acids are excised or inactivate the expression or function of the Pol gene.

In some embodiments, a first guide nucleic acid of the plurality of guide nucleic acids is complementary to a first target sequence in a Pro gene. In some embodiments, a second guide nucleic acid of the plurality of guide nucleic acids is complementary to a second target sequence in a Pro gene. In some embodiments, a third guide nucleic acid of the plurality of guide nucleic acid is complementary to a third target sequence in a Pro gene. In some embodiments, a fourth guide nucleic acid of the plurality of guide nucleic acid is complementary to a fourth target sequence in a Pro gene. In some embodiments, the first target sequence, the second target sequence, the third target sequence, and the fourth target sequence are different, wherein the intervening sequences between pairs of guide nucleic acids are excised or inactivate the expression or function of the Pro gene.

In some embodiments, a first guide nucleic acid of the plurality of guide nucleic acids is complementary to a first target sequence in a Env gene. In some embodiments, a second guide nucleic acid of the plurality of guide nucleic acids is complementary to a second target sequence in a Env gene. In some embodiments, a third guide nucleic acid of the plurality of guide nucleic acid is complementary to a third target sequence in a Env gene. In some embodiments, a fourth guide nucleic acid of the plurality of guide nucleic acid is complementary to a fourth target sequence in a Env gene. In some embodiments, the first target sequence, the second target sequence, the third target sequence, and the fourth target sequence are different, wherein the intervening sequences between pairs of guide nucleic acids are excised or inactivate the expression or function of the Env gene.

In some embodiments, a first guide nucleic acid of the plurality of guide nucleic acids is complementary to a first target sequence in a pX region gene. In some embodiments, a second guide nucleic acid of the plurality of guide nucleic acids is complementary to a second target sequence in a pX region gene. In some embodiments, a third guide nucleic acid of the plurality of guide nucleic acid is complementary to a third target sequence in a pX region gene. In some embodiments, a fourth guide nucleic acid of the plurality of guide nucleic acid is complementary to a fourth target sequence in a pX region gene. In some embodiments, the first target sequence, the second target sequence, the third target sequence, and the fourth target sequence are different, wherein the intervening sequences between pairs of guide nucleic acids are excised or inactivate the expression or function of the pX region gene.

In some embodiments, a first guide nucleic acid of the plurality of guide nucleic acids is complementary to a first target sequence in a HBZ or APH-2 gene. In some embodiments, a second guide nucleic acid of the plurality of guide nucleic acids is complementary to a second target sequence in a HBZ or APH-2 gene. In some embodiments, a third guide nucleic acid of the plurality of guide nucleic acid is complementary to a third target sequence in a HBZ or APH-2 gene. In some embodiments, a fourth guide nucleic acid of the plurality of guide nucleic acid is complementary to a fourth target sequence in a HBZ or APH-2 gene. In some embodiments, the first target sequence, the second target sequence, the third target sequence, and the fourth target sequence are different, wherein the intervening sequences between pairs of guide nucleic acids are excised or inactivate the expression or function of the HBZ or APH-2 gene.

In some embodiments, a first guide nucleic acid of the plurality of guide nucleic acids is complementary to a first target sequence in a Tax-1 gene. In some embodiments, a second guide nucleic acid of the plurality of guide nucleic acids is complementary to a second target sequence in a Tax-1 gene. In some embodiments, a third guide nucleic acid of the plurality of guide nucleic acid is complementary to a third target sequence in a Tax 1 gene. In some embodiments, a fourth guide nucleic acid of the plurality of guide nucleic acid is complementary to a fourth target sequence in a Tax-1 gene. In some embodiments, the first target sequence, the second target sequence, the third target sequence, and the fourth target sequence are different, wherein the intervening sequences between pairs of guide nucleic acids are excised or inactivate the expression or function of the Tax-1 gene.

In some embodiments, a first guide nucleic acid of the plurality of guide nucleic acids is complementary to a first target sequence in a Tax-2 gene. In some embodiments, a second guide nucleic acid of the plurality of guide nucleic acids is complementary to a second target sequence in a Tax-2 gene. In some embodiments, a third guide nucleic acid of the plurality of guide nucleic acid is complementary to a third target sequence in a Tax-2 gene. In some embodiments, a fourth guide nucleic acid of the plurality of guide nucleic acid is complementary to a fourth target sequence in a Tax-2 gene. In some embodiments, the first target sequence, the second target sequence, the third target sequence, and the fourth target sequence are different, wherein the intervening sequences between pairs of guide nucleic acids are excised or inactivate the expression or function of the Tax-2 gene.

In some embodiments, a composition comprises a combination of a plurality of guide nucleic acids targeting nucleic acid sequences of LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof, variants or orthologs thereof.

In some embodiments, a gRNA target sequence comprises a sequence at least or about 70%, 80%, 85%, 90% 91%, 92%, 93%, 94%, 95%, 96%, 97%, 98%, 99%, or 100% sequence identity to any one of SEQ ID NOs: 3, 4, 5 or 6. In some instances, a gRNA target sequence comprises a sequence at least or about 95% homology to any one of SEQ ID NOs: 3, 4, 5 or 6. In some instances, a gRNA target sequence comprises a sequence at least or about 95% homology to a sequence complementary to any one of SEQ ID NOs: 3, 4, 5 or 6. In some instances, a gRNA target sequence comprises a sequence at least or about 97% homology to any one of SEQ ID NOs: 3, 4, 5 or 6. In some instances, a gRNA target sequence comprises a sequence at least or about 97% homology to a sequence complementary to any one of SEQ ID NOs: 3, 4, 5 or 6. In some instances, a gRNA target sequence comprises a sequence at least or about 99% homology to any one of SEQ ID NOs: 3, 4, 5 or 6. In some instances, a gRNA target sequence comprises a sequence at least or about 99% homology to a sequence complementary to any one of SEQ ID NOs: 3, 4, 5 or 6. In some instances, a gRNA target sequence comprises a sequence at least or about 100% homology to any one of SEQ ID NOs: 3, 4, 5 or 6. In some instances, a gRNA target sequence comprises a sequence at least or about 100% homology to a sequence complementary to any one of SEQ ID NOs: 3, 4, 5 or 6.

In some embodiments, a gRNA target sequence comprises a sequence at least or about 70%, 80%, 85%, 90%, 91%, 92%, 93%, 94%, 95%, 96%, 97%, 98%, 99%, or 100% sequence identity to any one of SEQ TD NOs: 3, 4, 5 or 6. In some embodiments, a gRNA target sequence comprises a sequence at least or about 70%, 80%, 85%, 90%, 91%, 92%, 93%, 94%, 95%, 96%, 97% 98%, 99%, or 100% sequence identity to a sequence complementary to any one of SEQ ID NOs: 3, 4, 5 or 6. In some instances, a gRNA target sequence comprises a sequence at least or about 95% homology to any one of SEQ ID NOs: 3, 4, 5 or 6. In some instances, a gRNA target sequence comprises a sequence at least or about 95% homology to a sequence complementary to any one of SEQ ID NOs: 3, 4, 5 or 6. In some instances, a gRNA target sequence comprises a sequence at least or about 97% homology to any one of SEQ ID NOs: 3, 4, 5 or 6. In some instances, a gRNA target sequence comprises a sequence at least or about 97% homology to a sequence complementary to any one of SEQ ID NOs: 3, 4, 5 or 6. In some instances, a gRNA target sequence comprises a sequence at least or about 99% homology to any one of SEQ ID NOs: 3, 4, 5 or 6. In some instances, a gRNA target sequence comprises a sequence at least or about 99% homology to a sequence complementary to any one of SEQ ID NOs: 3, 4, 5 or 6. In some instances, a gRNA target sequence comprises a sequence at least or about 100% homology to any one of SEQ ID NOs: 3, 4, 5 or 6. In some instances, a gRNA target sequence comprises a sequence at least or about 100% homology to a sequence complementary to any one of SEQ ID NOs: 3, 4, 5 or 6 In some embodiments, a gRNA target sequence comprises a sequence at least or about 70%, 80%, 85%, 90%, 91%, 92%, 93%, 94%, 95%; 96%, 97%, 98%, 99%, or 100% sequence identity to any one of SEQ ID NOs: 1-6. In some instances, a gRNA target sequence comprises a sequence at least or about 95% homology to any one of SEQ ID NOs: 1-6. In some instances, a gRNA target sequence comprises a sequence at least or about 95% homology to a sequence complementary to any one of SEQ ID NOs: 1-6. In some instances, a gRNA target sequence comprises a sequence at least or about 97% homology to any one of SEQ ID NOs: 1-6. In some instances, a gRNA target sequence comprises a sequence at least or about 97% homology to a sequence complementary to any one of SEQ ID NOs: 1-6. In some instances, a gRNA target sequence comprises a sequence at least or about 99% homology to any one of SEQ ID NOs: 1-6. In some instances, a gRNA target sequence comprises a sequence at least or about 99% homology to a sequence complementary to any one of SEQ ID NOs: 1-6. In some instances, a gRNA target sequence comprises a sequence at least or about 100% homology to any one of SEQ ID NOs: 1-6. In some instances, a gRNA target sequence comprises a sequence at least or about 100% homology to a sequence complementary to any one of SEQ ID NOs: 1-6.

In some embodiments, a gRNA target sequence comprises a sequence at least or about 70%, 80%, 85%, 90%, 91%, 92%, 93%, 94%, 95%, 96%, 97%, 98%, 99%, or 100% sequence identity to any one of SEQ ID NOs: 1-6. In some embodiments, a gRNA target sequence comprises a sequence at least or about 70%, 80%, 85%, 90%, 91%, 92%, 93%, 94%, 95%, 96%, 97%, 98%, 99%, or 100% sequence identity to a sequence complementary to any one of SEQ ID NOs: 1-6. In some instances, a gRNA target sequence comprises a sequence at least or about 95% homology to any one of SEQ ID NOs: 1-6. In some instances, a gRNA target sequence comprises a sequence at least or about 95% homology to a sequence complementary to any one of SEQ ID NOs: 1-6. In some instances, a gRNA target sequence comprises a sequence at least or about 97% homology to any one of SEQ ID NOs: 1-6. In some instances, a gRNA target sequence comprises a sequence at least or about 97% homology to a sequence complementary to any one of SEQ ID NOs: 1-6. In some instances, a gRNA target sequence comprises a sequence at least or about 99% homology to any one of SEQ ID NOs: 1-6. In some instances, a gRNA target sequence comprises a sequence at least or about 99% homology to a sequence complementary to any one of SEQ ID NOs: 1-6. In some instances, a gRNA target sequence comprises a sequence at least or about 100% homology to any one of SEQ ID NOs: 1-6. In some instances, a gRNA target sequence comprises a sequence at least or about 100% homology to a sequence complementary to any one of SEQ ID NOs: 1-6.

In certain embodiments, a composition comprises a viral vector encoding a gene editing agent and at least one guide RNA (gRNA) wherein the gRNA is complementary to a target nucleic acid sequence of an HTLV gene sequence, comprising a coding and/or non-coding HTLV gene sequences. In certain embodiments, the guide nucleic acid sequences target one or more HTLV sequences comprising: structural gene sequence, enzymatic gene sequences, regulatory genes, accessory genes, transactivator gene sequences or combinations thereof. In certain embodiments, the guide nucleic acid sequences target one or more HTLV sequences comprising structural gene sequences, enzymatic gene sequences, regulatory genes, accessory genes, transactivator gene sequences or combinations thereof. In certain embodiments, HTLV target sequences comprise sequences in the LTR, Gag, Pol, Pro, Env, pX region, HBZ, APH-2, Tax-1, Tax-2 or combinations thereof. In certain embodiments, the gRNA is complementary to a long terminal repeat (LTR) of the HTLV.

In certain embodiments, a viral vector comprises an adenovirus vector, an adeno-associated viral vector (AAV), or derivatives thereof. In some embodiments, the nucleic acids are configured to be packaged into an adeno-associated virus (AAV) vector. In some embodiments, the adeno-associated virus (AAV) vector is AAV2, AAV5, AAV6, AAV7, AAV8, or AAV9. In some embodiments, the adeno-associated virus (AAV) vector is AAV1, AAV2, AAV3, AAV4, AAV5, AAV6, AAV7, AAV8, AAV9, AAV10, AAV11, AAVDJ, or AAVDJ/8.

In another embodiment, an expression vector comprises an isolated nucleic acid encoding a gene editing agent and at least one guide RNA (gRNA) wherein the gRNA is complementary to a target nucleic acid sequence of a retrovirus, e.g. HTLV gene sequence. In embodiments, the gene editing agents comprise: Cre recombinases, CRISPR/Cas molecules, TALE transcriptional activators, Cas9 nucleases, nickases, transcriptional regulators, homologues, orthologs or combinations thereof. In one embodiment, the gene editing agent is a Clustered Regularly Interspaced Short Palindromic Repeated (CRISPR)-associated endonuclease, or homologues or orthologs thereof. In another embodiment, the CRISPR-associated endonuclease is Cas9 or homologues or orthologs, thereof.

In some embodiments, the expression vector encodes a transactivating small RNA (tracrRNA) wherein the transactivating small RNA (tracrRNA) sequence is fused to the sequence encoding the guide RNA.

In another embodiment, the expression vector further comprises a sequence encoding a nuclear localization signal.

In some embodiments, the CRISPR-endonuclease is a Cas9 endonuclease, a Cas12 endonuclease, a CasX endonuclease, or a CasΦ endonuclease. In some embodiments, the CRISPR-endonuclease is a Cas9 nuclease. In some embodiments, the Cas9 nuclease is a Staphylococcus aureus Cas9 nuclease.

In some embodiments, the present disclosure provides a composition for the treatment or prevention of a retrovirus, e.g., HTLV, infection in a subject in need thereof. In some embodiments, the composition comprises at least one isolated guide nucleic acid comprising a nucleotide sequence that is complementary to a target region in LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequence, Tax-1 nucleic acid sequences, Tax-2 nucleic acid sequences or combinations thereof. In some embodiments, the composition comprises a CRISPR-associated (Cas) peptide, or functional fragment or derivative thereof. Together, the isolated nucleic acid guide molecule and the CRISPR-associated (Cas) peptide function to introduce one or more mutations or deletions at target sites within the LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof, which inhibit expression or function of these molecules thereby inhibiting infection by retrovirus, e.g., HTLV.

The composition also encompasses isolated nucleic acids encoding one or more elements of the CRISPR-Cas system. For example, in some embodiments, the composition comprises an isolated nucleic acid encoding at least one of the guide nucleic acid and a CRISPR-associated (Cas) peptide, or functional fragment or derivative thereof.

In some embodiments, the present disclosure provides a composition for the treatment or prevention of a retrovirus, e.g., HTLV infection in a subject in need thereof. In some embodiments, the composition comprises at least one isolated guide nucleic acid comprising a nucleotide sequence that is complementary to a target region in LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof. In some embodiments, the composition comprises a CRISPR-associated (Cas) peptide, or functional fragment or derivative thereof. Together, the isolated nucleic acid guide molecule and the CRISPR-associated (Cas) peptide function to introduce one or more mutations at target sites and/or excise the intervening sequences between two target sites, the target sites comprising LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof, which inhibit expression or function of these molecule thereby inhibiting infection by retroviruses, e.g., HTLV.

The composition also encompasses isolated nucleic acids encoding one or more elements of the CRISPR-Cas system. For example in some embodiments, the composition comprises an isolated nucleic acid encoding at least one of the guide nucleic acid and a CRISPR-associated (Cas) peptide, or functional fragment or derivative thereof.

In some embodiments, the present disclosure provides a method for the treatment or prevention of a retrovirus, e.g., HTLV infection in a subject in need thereof. In some embodiments, the method comprises administering to the subject an effective amount of a composition comprising at least one of a guide nucleic acid and a CRISPR-associated (Cas) peptide, or functional fragment or derivative thereof. In certain instances the method comprises administering a composition comprising an isolated nucleic acid encoding at least one of the guide nucleic acid and a CRISPR-associated (Cas) peptide, or functional fragment or derivative thereof. In certain embodiments, the method comprises administering a composition described herein to a subject diagnosed with a retrovirus infection, e.g., HTLV, at risk for developing a retrovirus infection, e.g., HTLV, and the like.

Gene Editing Agents

Compositions of the disclosure include at least one gene editing agent, comprising CRISPR-associated nucleases such as Cas9 and Cas12a gRNAs, Argonauts family of endonucleases, clustered regularly interspaced short palindromic repeat (CRISPR) nucleases, zinc-linger nucleases (ZFNs), transcription activator-like effector nucleases (TALENs), meganucleases, other endo- or exo-nucleases, or combinations thereof.

In recent years, several systems for targeting endogenous genes have been developed including homing endonucleases (HE) or meganucleases, zinc finger nucleases (ZFN), transcription activator-like effector nucleases (TALEN) and most recently clustered regularly interspaced short palindromic repeats (CRISPR)-associated system 9 (Cas9) proteins which utilize site-specific double-strand DNA break (DSB)-mediated DNA repair mechanisms. These enzymes induce a precise and efficient genome cutting through DSB-mediated DNS repair mechanisms. These DSB-mediated genome editing techniques enable target gene deletion, insertion, or modification.

In the past years, ZFNs and TALENs have revolutionized genome editing. The major drawbacks for ZFNs and TALENs are the uncontrollable of target effects and the tedious and expensive engineering of custom DNA-binding fusion protein for each target site, which limit the universal application and clinical safety.

CRISPR (Clustered Regularly Interspaced Short Palindromic Repeats) is found in bacteria and is believed to protect the bacteria front phase infection. It has recently been used as a means to alter gene expression in eukaryotic DNA, but has not been proposed as an anti-viral therapy or more broadly as a way to disrupt genomic material. Rather, it has been used to introduce insertions or deletions as a way of increasing or decreasing transcription in the DNA of a targeted cell or population of cells. See for example, Horvath et al., Science (2010) 327:167-170; Terns et al., Current Opinion in Microbiology (2011) 14:321-327; Bhaya et al., Annu Rev Genet (2011) 45:273-297; Wiedenheft et al., Nature (2012) 482:331-338); Jinek M et al., Science (2012) 337:816-821; Cong L et al., Science (2013) 339:819-823; Jinek M et al., (2013) eLife 2:e00471; Mali P et al. (2013) Science 339:823-826; Qi L S et al. (2013) Cell 152:1173-1183; Gilbert L A et al. (2013) Cell 154:442451; Yang H et al. (2013) Cell 154:1370-1379; and Wang H et al. (2013) Cell 153:910-918).

CRISPR methodologies employ a nuclease, CRISPR-associated (Cas), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas and guide RNA (gRNA) may be synthesized by known methods. Cas/guide-RNA (gRNA) uses a non-specific DNA cleavage protein Cas, and an RNA oligonucleotide to hybridize to target and recruit the Cas/gRNA complex. See Chang et al., 2013, Cell. Res. 23:465-472; Hwang et al., 2013, Nat. Biotechnol. 31:227-229; Xiao et al., 2013, Nucl. Acids Res. 1-11.

In general, the CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNase domains, protein-protein interaction domains, dimerization domains, as well as other domains. The mechanism through which CRISPR/Cas9-induced mutations inactivate the provirus can vary. For example, the mutation cat affect proviral replication, and viral gene expression. The mutation can comprise one or more deletions. The size of the deletion can vary from a single nucleotide base pair to about 10,000 base pairs. In some embodiments, the deletion can include all or substantially all of the proviral sequence. In some embodiments the deletion can eradicate the provirus. The mutation can also comprise one or more insertions, that is, the addition of one or more nucleotide base pairs to the proviral sequence. The size of the inserted sequence also may vary, for example from about one base pair to about 300 nucleotide base pairs. The mutation can comprise one or more point mutations, that is, the replacement of a single nucleotide with another nucleotide. Useful point mutations are those that have functional consequences, for example, mutations that result in the conversion of an amino acid codon into a termination, codon, or that result in the production of a nonfunctional protein.

CRISPR methodologies employ a nuclease, CRISPR-associated (Cas), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas and guide RNA (gRNA) may be synthesized by known methods. Cas/guide-RNA (gRNA) uses a non-specific DNA cleavage protein Cas, and an RNA oligonucleotide to hybridize to target and recruit the Cas/gRNA complex. See Chang et al., 2013, Cell Res. 23:465-472; Hwang et al., 2013, Nat. Biotechnol. 31:227-229; Xiao et al., 2013, Nucl. Acids Res. 1-11.

The RNA-guided Cas9 biotechnology induces genome editing without detectable off-target effects. This technique takes advantage of the genome defense mechanisms in bacteria that CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR (Clustered Regularly interspaced Short Palindromic Repeats) RNA (crRNA). Cas9 belongs to the type II CRISPR/Cas system and has strong endonuclease activity to cut target DNA.

In certain embodiments, the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein.

In some embodiments, the CRISPR/Cas-like protein can be derived from a wild type Cas9 protein or fragment thereof. In other embodiments, the CRISPR/Cas-like protein can be derived from modified Cas9 protein. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

In one embodiment, the RNA-guided endonuclease is derived from a type II CRISPR/Cas system. The CRISPR-associated endonuclease, Cas9, belongs to the type II CRISPR/Cas system and has strong endonuclease acuity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately. Therefore, the Cas9 gRNA technology requires the expression of the Cas9 protein and gRNA, which then form a gene editing complex at the specific target DNA binding site within the target genome and inflict cleavage/mutation of the target DNA.

However, the present disclosure is not limited to the use of Cas9-mediated gene editing. Rather, the present disclosure encompasses the use of other CRISPR-associated peptides, which can be targeted to a targeted sequence using a gRNA and can edit to target site of interest. For example, in some embodiments, the disclosure utilizes Cas12a (also known as Cpf1) to edit the target site of interest.

Engineered CRISPR systems generally contain two components: a guide RNA (gRNA or sgRNA) and a CRISPR-associated endonuclease (Cas protein). In nature, CRISPR/CRISPR-associated (Cas) systems provide bacteria and archaea with adaptive immunity against viruses and plastids by using CRISPR RNAs (crRNAs) to guide the silencing of invading nucleic acids. The CRISPR-Cas is a RNA-mediated adaptive defense system that relies on small RNA molecules for sequence-specific detection and silencing of foreign nucleic acids. CRISPR/Cas systems are composed of cas genes organized in operon(s) and CRISPR array(s) consisting of genome-targeting sequences (called spacers).

As described herein. CRISPR-Cas systems generally refer to an enzyme system that includes a guide RNA sequence that, contains a nucleotide sequence complementary or substantially complementary to a region of a target polynucleotide, and a protein with nuclease activity. CRISPR-Cos systems include Type I CRISPR-Cos system, Type II CRISPR-Cas system, Type III CRISPR-Cas system, and derivatives thereof. CRISPR-Cas systems include engineered and/or programmed nuclease systems derived from naturally accruing CRISPR-Cas systems. In certain embodiments, CRISPR-Cas systems contain engineered and/or mutated Cas proteins. In some embodiments, nucleases generally refer to enzymes capable of cleaving the phosphodiester bonds between the nucleotide subunits of nucleic acids. In some embodiments, endonucleases are generally capable of cleaving the phosphodiester bond within a polynucleotide chain. Nickases refer to endonucleases that cleave only a single strand of a DNA duplex.

In some embodiments, the CRISPR/Cas system used herein can be a type I, a type II, or a type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, CasX, CasΦ, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. By way of further example, in some embodiments, the CRISPR-Cas protein is a Cas1, Cas1B, Cas2, Cas3, Cas4, Cas5, Cash, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx16, CsaX, Csx3, Csx1, Csx15, Csf1, Csf2, Csf3, Csf4, Cas9, Cas12 (e.g., Cas12a, Cas12b, Cas12c, Cas12d, Cas12k, Cas12j/CasΦ, Cas12L etc.), Cas13 (e.g., Cas13a, Cas13b (such as Cas13b-t1, Cas13b-t2, Cas13b-t3), Cas13c, Cas13d, etc.), Cas14, CasX, CasY, or an engineered form of the Cas protein. In some embodiments, the CRISPR/Cas protein or endonuclease is Cas9. In some embodiments, the CRISPR/Cas protein or endonuclease is Cas12. In certain embodiments, the Cas12 poly peptide is Cas12a, Cas12b, Cas12c, Cas12d, Cas12e, Cas12g, Cas12h, Cas12i, Cas12L or Cas12J. In some embodiments, the CRISPR/Cas protein or endonuclease is CasX. In some embodiments, the CRISPR/Cas protein or endonuclease is CasY. In some embodiments, the CRISPR/Cas protein or endonuclease is CasΦ.

In some embodiments, the Cas9 protein can be from or derived from: Staphylococcus aureus, Streptococcus pyogenes, Streptococcus thermophilus, Streptococcus sp., Nocardiopsis dassonvillet, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus, selenitireducens, Exiguobacterium sibiricum, Lactobacillus delbrueckii, Lactobacillus salivarius, Microscilla marina, Burkholderiales bacterium, Polaromonas naphthalenivorims, Polaromonas sp., Crocosphaera warsonii, Cyanothece sp., Microcystis aeruginosa, Synechococcus sp., Acetohalobium arabaticum, Ammonifex degensii, Caldicelulosiruptor becseii, Candidatus desulforudis, Clostridium botulinum, Clostridium difficile, Fine goldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

In some embodiments, the composition comprises a CRISPR-associated (Cas) protein, or functional fragment or derivative thereof. In some embodiments, the Cas protein is an endonuclease, including but not limited to the Cas9 nuclease. In some embodiments, the Cas9 protein comprises an amino acid sequence identical to the wild type Streptococcus pyogenes or Staphylococcus aureus Cas9 amino acid sequence. In some embodiments, the Cas protein comprises the amino acid sequence of a Cas protein from other species, for example other Streptococcus species, such as thermophilus; Pseudomonas aeruginosa, Escherichia coli, or other sequenced bacteria genomes and archaea, or other prokaryotic microorganisms. Other Cas proteins, useful for the present disclosure, known or can be identified, using methods known in the art (see e.g., Esvelt et al, 2013, Nature Methods, 10: 1116-1121). In some embodiments, the Cas protein comprises a modified amino acid sequence, as compared to its natural source. CRISPR/Cas proteins comprise at least one RNA recognition and/or RNA binding domain. RNA recognition and/or RNA binding domains interact with guide RNAs (gRNAs). CRISPR/Cas proteins can also comprise nuclease domains (i.e., DNase or RNase domains), DNA binding domains, helicase domains, RNAse domains, protein-protein interaction domains, dimerization domains, as well as other domains.

The CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the Cas protein. The CRISPR/Cas-like protein can also be truncated or modified to optimize the activity of the effector domain of the Cas protein.

In some embodiments, the CRISPR/Cas-like protein can be derived from a wild type Cas protein or fragment thereof. In some embodiments, the CRISPR/Cas-like protein is a modified Cas9 protein. For example, the amino acid sequence of the Cas9 protein can be modified to alter one or more properties (e.g., nuclease activity, affinity, stability, etc.) of the protein relative to wild-type or another Cas protein. Alternatively, domains of the Cas9 protein not involved in RNA-guided cleavage can be eliminated from the protein such that the modified Cas9 protein is smaller than the wild-type Cas9 protein.

The disclosed CRISPR-Cas compositions should also be construed to include any fumy of a protein having substantial homology to a Cas protein (e.g., Cas9, saCas9, Cas9 protein) disclosed herein. In some embodiments, a protein which is “substantially homologous” is about 50% homologous, about 70% homologous, about 80% homologous, about 90% homologous, about 95% homologous, or about 99% homologous to amino acid sequence of a Cas protein disclosed herein. The Cas9 can be an orthologous. Six smaller Cas9 orthologues have been used and reports have shown that Cas9 from Staphylococcus aureus (SaCas9) can edit the genome with efficiencies similar to those of SpCas9, wile being more than 1 kilobase shorter.

In some embodiments, the composition comprises a CRISPR-associated (Cas) peptide, or functional fragment or derivative thereof. In certain embodiments, the Cas peptide is an endonuclease, including but not limited to the Cas9 nuclease. In some embodiments, the Cas9 peptide comprises an amino acid sequence identical to the wild type Streptococcus pyogenes Cas9 amino acid sequence. In some embodiments, the Cas peptide may comprise the amino acid sequence of a Cas protein from other species, for example other Streptococcus species, such as thermophilus; Pseudomonas aeruginosa, Escherichia coli, or other sequenced bacteria genomes and archaea, or other prokaryotic microorganisms. Other Cas peptides, useful for the present disclosure, known or can be identified, using methods known in the art (see e.g., Esvelt et al., 2013, Nature Methods, 10: 1116-1121). In certain embodiments, the Cas peptide may comprise a modified amino acid sequence, as compared to its natural source. For example, in some embodiments, the wild type Streptococcus pyogenes Cas9 sequence can be modified. In certain embodiments, the amino acid sequence can be codon optimized for efficient expression in human cells (i.e., “humanized) or in a species of interest. A humanized Cas9 nuclease sequence can be for example, the Cas9 nuclease sequence encoded by any of the expression vectors listed in Genbank accession numbers KM099231.1 GL669193757; KM099232.1 GL669193761; or KM099233.1 GL669193765. Alternatively, the Cas9 nuclease sequence can be for example, the sequence contained within a commercially available vector such as PX330 or PX260 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GL669103757; KM099232.1 GL669193761; or KM099233.1 GL669193765 or Cas9 amino acid sequence of PX330 or PX260 (Addgene, Cambridge, Mass.).

The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution).

In certain embodiments, the Cas peptide is a mutant Cas9, wherein the mutant Cas9 reduces the off-target effects, as compared to wild-type Cas9. In some embodiments, the mutant Cas9 is a Streptococcus pyogenes Cas9 (SpCas9) variant.

In some embodiments, SpCas9 variants comprise one or more point mutations, including, but not limited to R780A, K810A, K848A, K855A, H982A, K1003A, and R1060A (Slaymaker et al., 2016, Science, 351(6268); 84-88). In some embodiments, SpCas9 variants comprise D1135E point mutation (Kleinstiver et al., 2015, Nature, 523(7561): 481-485). In some embodiments, SpCas9 variants comprise one or more point mutations, including, but not limited to N497A, R661A, Q695A, Q926A, D1135E, L169A, and Y450A (Kleinstiver et al., 2016, Nature, doi:10.1038/nature16526). In some embodiments, SpCas9 variants comprise one or more point mutations, including but not limited to M495A, M694A, and M698A. Y450 is involved with hydrophobic base pair stacking. N497, R661, Q695, Q926 are involved with residue to base hydrogen bonding contributing to off-target effects. N497 hydrogen bonding through peptide backbone. L169A is involved with hydrophobic base pair stacking. M495A, M694A, and H698A are involved with hydrophobic base pair stacking.

In some embodiments, SpCas9 variants comprise one or more point mutations at one or more of the following residues: R780, K810, K848, K855, H982, K1003, R1060, D1135, N497, R661, Q695, Q926, L169, Y450, M495, M694, and M698. In some embodiments, SpCas9 variants comprise one or more point mutations selected from the group of: R780A, K810A, K848A, K855A, H982A, K1003A, R1060A, D1135E, N497A, R661A, Q695A, Q926A, L169A, Y450A, M495A, M694A, and M698A.

In some embodiments, the SpCas9 variant comprises the point mutations, relative to wildtype SpCas9, of N497A, R661A, Q695A, and Q926A. In some embodiments, the SpCas9 variant comprises the point mutations, relative to wildtype SpCas9, of N497A, R661A, Q695A, Q926A, and D1135E. In some embodiments, the SpCas9 variant comprises the point mutations, relative to wildtype SpCas9, of N497A, R661A, Q695A, Q926A, and L169A. In some embodiments, the SpCas9 variant comprises the point mutations, relative to wildtype SpCas9, of N497A, R661A, Q695A, Q926A, and Y450A. In some embodiments, the SpCas9 variant comprises the point mutations, relative to wildtype SpCas9, of N497A, R661A, Q695A, Q926A, and M495A. In some embodiments, the SpCas9 variant comprises the point mutations, relative to wildtype SpCas9, of N497A, R661A, Q695A, Q926A, and M694A. In some embodiments, the SpCas9 variant comprises the point mutations, relative to wildtype SpCas9, of N497A, R661A, Q695A, Q926A, and H698A. In some embodiments, the SpCas9 variant comprises the point mutations, relative to wildtype SpCas9, of N497A, R661A, Q695A, Q926A, D1135E, and L169A. In some embodiments, the SpCas9 variant comprises the point mutations, relative to wildtype SpCas9, of N497A, R661A, Q695A, Q926A, D1135E, and Y450A. In some embodiments, the SpCas9 variant comprises the point mutations, relative to wildtype SpCas9, of N497A, R661A, Q695A, Q926A, D1135E, and M495A. In some embodiments, the SpCas9 variant comprises the point mutations, relative to wildtype SpCas9, of N497A, R661A, Q695A, Q926A, D1135E, and M694A. In some embodiments, the SpCas9 variant comprises the point mutations, relative to wildtype SpCas9, of N497A, R661A, Q695A, Q926A, D1135E, and M698A.

In some embodiments, the SpCas9 variant comprises the point mutations, relative to wildtype SpCas9, of R661A, Q695A, and Q926A. In some embodiments, the SpCas9 variant comprises the point mutations, relative to wildtype SpCas9, of R661A, Q695A, Q926A, and D1135E. In some embodiments, the SpCas9 variant comprises the point mutations, relative to wildtype SpCas9, of R661A, Q695A, Q926A, and L169A. In some embodiments, the SpCas9 variant comprises the point mutations, relative to wildtype SpCas9, of R661A, Q695A, Q926A, and Y450A. In some embodiments, the SpCas9 variant comprises the point mutations, relative to wildtype SpCas9, of R661A, Q695A, Q926A, and M495A. In some embodiments, the SpCas9 variant comprises the point mutations, relative to wildtype SpCas9, of R661A, Q695A, Q926A, and M694A. In some embodiments, the SpCas9 variant comprises the point mutations, relative to wildtype SpCas9, of R661A, Q695A, Q926A, and H698A. In some embodiments, the SpCas9 variant comprises the point mutations, relative to wildtype SpCas9, of R661A, Q695A, Q926A, D1135E, and L169A. In some embodiments, the SpCas9 variant comprises the point mutations, relative to wildtype SpCas9, of R661A, Q695A, Q926A, D1135E, and Y450A. In some embodiments, the SpCas9 variant comprises the point mutations, relative to wildtype SpCas9, of R661A, Q695A, Q926A, D1135E, and M495A. In some embodiments, the SpCas9 variant comprises the point mutations, relative to wildtype SpCas9, of R661A, Q695A, Q926A, D1135E, and M694A. In some embodiments, the SpCas9 variant comprises the point mutations, relative to wildtype SpCas9, of R661A, Q695A, Q926A, D1135E, and M698A.

In some embodiments, the mutant Cas9 comprises one or more mutations that alter PAM specificity (Kleinstiver et al., 2015, Nature, 523(7561):481-485; Kleinstiver et al., 2015, Nat Biotechnol, 33(12): 1293-1298). In some embodiments, the mutant Cas9 comprises one or more mutations that alter the catalytic activity of Cas9, including but not limited to D10A in RuvC and H840A in HNH (Cong et al., 2013; Science 339: 919-823, Gasiubas et al., 2012; PNAS 109:E2579-2586 Jinek et al., 2012, Science 337: 816-821).

In addition to the wild type and variant Cas9 endonucleases described, embodiments of the disclosure also encompass CRISPR systems including newly developed “enhanced-specificity” S. pyogenes Cas9 variants (eSpCas9), which dramatically reduce off target cleavage. These variants are engineered with alanine substitutions to neutralize positively charged sites in a groove that interacts with the non-target strand of DNA. This aim of this modification is to reduce interaction of Cas9 with the non-target strand, thereby encouraging re-hybridisation between target and non-target strands. The effect of this modification is a requirement far more stringent Watson-Crick pairing between the gRNA and the target DNA strand, which limits off-target cleavage (Slaymaker, L. M. et al. (2015) DOI: 10.1126/science.aad5227).

In certain embodiments, three variants found to have the best cleavage efficiency and fewest off-target effects: SpCas9 (K855A), SpCas9 (K810A/K1003A/R1060A) (a.k.a. eSpCas9 1.0), and SpCas9(K848A/K1003A/R1060A) (a.k.a. eSPCas9 1.1) are employed in the compositions. The disclosure is by no means limited to these variants, and also encompasses all Cas9 variants (Slaymaker, I. M. et al. (2015)). The present disclosure also includes another type of enhanced specificity Cas9 variant, “high fidelity” spCas9 variants (HF-Cas9). Examples of high fidelity variants include SpCas9-HF1 (N497A/R661A/Q695A/Q926A), SpCas9-HF2 (N497A/R661A/Q695A/Q926A/D1135E), SpCas9-HF3 (N497A/R661A/Q695A/Q926A/L169A), SpCas9-HF4 (N497A/R661A/Q695A/Q926A/Y450A). Also included are all SpCa9 variants bearing all possible single, double, triple and quadruple combinations of N497A, R661A, Q695A, Q926A or any other substitutions (Kleinstiver. B. P. et al., 2016, Nature. DOI: 10.1038/nature16526).

Accordingly, in certain embodiments, a Cas9 variant comprises a human-optimized Cas9, a nickase mutant Cas9; saCas9; enhanced-fidelity SaCas9 (efSaCas9); SpCas9(K855a); SpCas9(K810A/K1003A/r1060A); SpCas9(K848A/K1003A/R1060A); SpCas9 N497A, R661A, Q695A, Q926A; SpCas9 N497A, R661A, Q695A, Q926A, D1135E; SpCas9 N497A, R661A, Q695A, Q926A L169A; SpCas9 N497A, R661A, Q695A, Q926A Y450A; SpCas9 N497A, R661A, Q695A, Q926A M495A; SpCas9 N497A, R661A, Q695A, Q926A M694A; SpCas9 N497A, R661A, Q695A, Q926A H698A; SpCas9 N497A, R661A, Q695A, Q926A, D1135E, L169A; SpCas9 N497A, R661A, Q695A, Q926A, D1135E, Y450A; SpCas9 N497A, R661A, Q695A, Q926A, D1135E, M495A; SpCas9 N497A, R661A, Q695A, Q926A, D1135E, M694A; SpCas9 N497A, R661A, Q695A, Q926A, D1135E, M698A; SpCas9 R661A, Q695A, Q926A; SpCas9 R661A, Q695A, Q926A, D1135E, SpCas9 R661A, Q695A, Q926A, L169A; SpCas9 R661A, Q695A, Q926A Y450A; SpCas9 R661A, Q695A, Q926A M495A; SpCas9 R661A, Q695A, Q926A M694A, SpCas9 R661A, Q695A, Q926A H698A; SpCas9 R661A, Q695A, Q926A D1135E L169A; SpCas9 R661A, Q695A, Q926A D1135E Y450A; SpCas9 R661A, Q695A, Q926A D1135E M495A; or SpCas9 R661A, Q695A, Q926A, D1135E or M694A.

As used herein, the term “Cas” is meant to include all Cas molecules comprising variants, mutants, orthologues, high-fidelity variants and the like.

However, the present disclosure is not limited to the use of Cas9-mediated gene editing. Rather, the present disclosure encompasses the use of other CRISPR-associated peptides, which can be targeted to a targeted sequence using a gRNA and can edit to target site of interest. For example, in some embodiments, the disclosure utilizes Cpf1 to edit the target site of interest. Cpf1 is a single crRNA-guided, class 2 CRISPR effector protein which can effectively edit target DNA sequences in human cells. Exemplary Cpf1 includes, but is not limited to, Acidaminococcus sp. Cpf1 (AsCpf1) and Lachnospiraceae bacterium Cpf1 (LbCpf1).

The disclosure should also be construed to include any form of a peptide having substantial homology to a Cas peptide (e.g., Cas9) disclosed herein. Preferably, a peptide which is “substantially homologous” is about 50% homologous, more preferably about 70% homologous, even more preferably about 80% homologous, more preferably about 90% homologous, even more preferably, about 93% homologous, and even more preferably about 99% homologous to amino acid sequence of a Cas peptide disclosed herein.

The peptide may alternatively be made by recombinant means or by cleavage from a longer polypeptide. The composition of a peptide may be confirmed by amino acid analysis or sequencing.

The variants of the peptides according to the present disclosure may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups. (iii) one in which the peptide is an alternative splice variant of the peptide of the present disclosure. (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one polypeptide to a sequence of a second polypeptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The present disclosure includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The peptides of the disclosure can be post-translational) modified. For example, post-translational modifications that fall within the scope of the present disclosure include signal peptide cleavage, glycosylation, acetylation, isoprenylation, proteolysis, myristylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the disclosure may include unnatural amino acids formed by post-translational modification or by introducing unnatural amino acids during translation. A variety of approaches are available for introducing unnatural amino acids during protein translation.

A peptide or protein of the disclosure may be conjugated with other molecules, such as proteins, to prepare fusion proteins. This may be accomplished, for example, by the synthesis of N-terminal or C-terminal fusion proteins provided that the resulting fusion protein retains the functionality of the Cas peptide.

A peptide or protein of the disclosure may be phosphorylated using conventional methods such as the method described in Reedijk et al. (The EMBO Journal 11(4):1365, 1992).

Cyclic derivatives of the peptides of the disclosure are also part of the present disclosure. Cyclization may allow the peptide to assume a more favorable conformation for association with other molecules. Cyclization may be achieved using techniques known in the art. For example, disulfide bonds may be formed between two appropriately spaced components having free sulfydryl groups, or an amide bond may be formed between an amino group of one component and a carboxyl group of another component. Cyclization may also be achieved using an azabenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the disclosure, cyclic peptides may comprise a beta-turn in the right position. Beta-turns may be introduced into the peptides of the disclosure by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulfide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The disclosure also relates to peptides comprising a Cas peptide fused to, or integrated into, a target protein, and/or a targeting domain capable of directing the chimeric protein to a desired cellular component or cell type or tissue. The chimeric proteins may also contain additional amino acid sequences or domains. The chimeric proteins are recombinant in the sense that the various components are from different sources, and as such are not found together in nature (i.e. are heterologous).

In some embodiments, the targeting domain can be a membrane spanning domain, a membrane binding domain, or a sequence directing the protein to associate with for example vesicles or with the nucleus. In some embodiments, the targeting domain can target a peptide to a particular cell type or tissue. For example, the targeting domain can be a cell surface ligand or an antibody against cell surface antigens of a target tissue (e.g. cancerous tissue). A targeting domain may target the peptide of the disclosure to a cellular component. In certain embodiments, the targeting domain targets a tumor-specific antigen or tumor-associated antigen.

N-terminal or C-terminal fusion proteins comprising a peptide or chimeric protein of the disclosure conjugated with other molecules may be prepared by fusing, through recombinant techniques, the N-terminal or C-terminal of the peptide or chimeric protein, and the sequence of a selected protein or selectable marker with a desired biological function. The resultant fusion proteins contain the Cas peptide or chimeric protein fused to the selected protein or marker protein as described herein. Examples of proteins which may be used to prepare fusion proteins include immunoglobulins, glutathione-S-transferase (GST), hemagglutinin (HA), and truncated myc.

A peptide of the disclosure may be synthesized by conventional techniques. For example, the peptides of the disclosure may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford, Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis.).

A peptide of the disclosure may be prepared by standard chemical or biological means of peptide synthesis. Biological methods include, without limitation, expression of a nucleic acid encoding a peptide in a host cell or in an in vitro translation system.

Biological preparation of a peptide of the disclosure involves expression of a nucleic acid encoding a desired peptide. An expression cassette comprising such a coding sequence may be used to produce a desired peptide. For example, subclones of a nucleic acid sequence encoding a peptide of the disclosure can be produced using conventional molecular genetic manipulation for subcloning gene fragments, such as described by Sambrook et al., Molecular Cloning: A Laboratory Manual, Cold Springs Laboratory, Cold Springs Harbor, New York (2012), and Ausubel et al. (ed.), Current Protocols in Molecular Biology, John Wiley & Sons (New York, N.Y.) (1999 and preceding editions), each of which is hereby incorporated by inference in its entirety. The subclones then are expressed in vitro or in vivo in bacterial cells to yield a smaller protein or polypeptide that can be tested for a particular activity.

In the context of an expression vector, the vector can be readily introduced into a host cell, e.g., mammalian, bacterial, yeast or insect cell by any method in the art. Coding sequences for a desired peptide of the disclosure may be codon optimized based on the codon usage of the intended host cell in order to improve expression efficiency as demonstrated herein. Codon usage patterns can be found in the literature (Nakamura et al., 2000, Nuc. Acids Res. 28:292). Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The expression vector can be transferred into a host cell by physical, biological or chemical means, discussed in detail elsewhere herein.

To ensure that the peptide obtained from either chemical or biological synthetic techniques is the desired peptide, analysis of the peptide composition can be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

The peptides and chimeric proteins of the disclosure may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

In certain embodiments, a gene editing system comprises meganucleases. In some embodiments, the gene editing system comprises zinc finger nucleases (ZFNs). In some embodiments, the gene editing system comprises transcription activator-like effector nucleases (TALENs). These gene editing systems can be broadly classified into two categories based on their mode of DNA recognition: ZFNs, TALENs and meganucleases achieve specific DNA binding via protein DNA interactions, whereas CRISPR-Cas systems are targeted to specific DNA sequences by a short RNA mode molecule that base-pairs directly with the target DNA and by protein-DNA interactions. Accordingly, protein targeting or nucleic acid targeting can be employed to target Lilt nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof.

Guide Nucleic Acids

The compositions of the disclosure include sequence encoding a guide RNA (gRNA) comprising a sequence that is complementary to a target sequence in aretrovirus. e.g. HTLV.

In some embodiments, the composition comprises at least one isolated guide nucleic acid, or fragment thereof, where the guide nucleic acid comprises a nucleotide sequence that is complementary to one or more target sequences in the genes encoding LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof. In some embodiments, the guide nucleic acid is a guide RNA (gRNA).

In some embodiments, the gRNA comprises a crRNA:tracrRNA duplex. In some embodiments, the gRNA comprises a stem-loop that mimics the natural duplex between the crRNA and tracrRNA. In some embodiments, the stem-loop comprises a nucleotide sequence comprising AGAAAU. For example in some embodiments, the composition comprises a synthetic or chimeric guide RNA comprising a crRNA, stem, and tracrRNA.

In certain embodiments, the composition comprises an isolated crRNA and/or an isolated tracrRNA which hybridize to form a natural duplex. For example, in some embodiments, the gRNA comprises a crRNA or crRNA precursor (pre-crRNA) comprising a targeting sequence.

In some embodiments, the gRNA comprises a nucleotide sequence that is substantially complementary to a target sequence in the genes encoding LTR nucleic acid sequences, Gag nucleic acid sequencers, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof. The target sequence may be any sequence in any coding or non-coding region where CRISPR/Cas-mediated gene editing would result in the mutation of the genome and inhibition of viral infectivity. In certain embodiments, the target sequence, to which the gRNA is substantially complementary, is within the gene sequences encoding LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof.

Exemplary gRNA nucleotide sequences for targeting LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof, comprise sequences targeting or hybridizing to SEQ ID NOS: 1-6 or to the complementary sequences thereof. In certain embodiments, the gRNA nucleotides comprise sequences targeting or hybridizing to SEQ ID NOS: 1-6 or to the complementary sequences thereof. Further, the disclosure encompasses an isolated nucleic acid (e.g., gRNA) having substantial homology to a nucleic acid disclosed herein. In certain embodiments, the isolated nucleic acid has at least 75%, 80%, 85%, 90%, 95%, 96%, 97%, 98%, or 99% sequence homology with a nucleotide sequence of a gRNA described elsewhere herein.

The guide RNA sequence can be a sense or anti-sense sequence. The guide RNA sequence generally includes a proto-spacer adjacent motif (PAM). The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologs may have different PAM specificities. For example, Cas9 from S. thermophilus requires 5′-NNAGAA for CRISPR 1 and 5′-NGGNG for CRISPR3) and Neisseria meningitidis requires 5′-NNNNGATT). The specific sequence of the guide RNA may vary, but, regardless of the sequence, useful guide RNA sequences will be those that minimize off-target effects while achieving high efficiency and complete ablation of the HTLV. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides. Useful selection methods identify regions having extremely low homology between the foreign viral genome and host cellular genome including endogenous retroviral DNA, include bioinformatic screening using 12-bp+NGG target-selection criteria to exclude of target human transcriptome or (even rarely) untranslated-genomic sites.

The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs.

When the compositions are administered in an expression vector, the guide RNAs can be encoded by a single vector. Alternatively, multiple vectors can be engineered to each include two or more different guide RNAs. Useful configurations will result in the excision of viral sequences between cleavage sites resulting in the ablation of HTLV genome or HTLV protein expression. Thus, the use of two or more different guide RNAs promotes excision of the viral sequences between the cleavage sites recognized by the CRISPR endonuclease. The excised region can van in size from a single nucleotide to several thousand nucleotides. Exemplary excised regions are described herein.

When the compositions are administered as a nucleic acid or are contained within an expression vector, the CRISPR endonuclease can be encoded by the same nucleic acid or vector as the guide RNA sequences. Alternatively, or in addition, the CRISPR endonuclease can be encoded in a physically separate nucleic acid from the guide RNA sequences or in a separate vector.

In some embodiments, the RNA molecules e.g. crRNA, tracrRNA, gRNA are engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington D.C.). Modified RNA components include the following: 2′-O-methylcytidine; N⁴-methylcytidine; N⁴-2′-O-dimethylcytidine; N⁴-acetylcytidine; 5-methylcytidine; 5,2′-O-di methylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine 3,2′-O-dimethylurdine; 3-(3-amino-3-carboxypropyl)uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethyl-2′-thiouridine, 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-O-methyl-uridine; 5-carboxymethylaminomethyl-2-thiouridine; dihydrouridine; dihydroribosylthymine; 2′-methyladenosine; 2-methyladenosine; N⁶N-methyladenosine; N⁶,N⁶-dimethyladenosine; N⁶,2′-O-trimethyladenosine; 2-methylthio-N⁶N-isopentenyladenosine, N⁶-(cis-hydroxyisopentenyl)-adenosine; 2-methylthio-N⁶-(cis-hydroxyisopentenyl)-adenosine; N⁶-glycinylcarbamoyl)adenosine; N⁶-threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio N⁶-hydroxnorvalylcarbamoyl adenosine; 2′-O-ribosyladenosine (phosphate); inosine; 2′O-methyl inosine; 1-methyl inosine; 1;2′-O-dimethyl inosine; 2′-O-methyl guanosine; 1-methyl guanosine; N²-methyl guanosine; N²,N²-dimethyl guanosine; N²,2′-O-dimethyl guanosine; N²,N²,2′-O-trimethyl guanosine; 2′-O-ribosyl guanosine (phosphate); 7-methyl guanosine; N²;7-dimethyl guanosine; N²; N²;7-trimethyl guanosine; wyosine; methylwyosine; under-modified hydroxywybutosine; wybutosine; hydroxywybutosine; peroxywybutosine; queuosine; epoxyqueuosine, galactosyl-queuosine; mannosyl-queuosine; 7-cyan-7-deazaguanosine; arachaeosine [also called 7-formamido-7-deazaguanosine]; and 7-aminomethyl-7-deazaguanosine.

In certain embodiments, the composition comprises multiple different gRNAs, each targeted to a different target sequence. In certain embodiments, this multiplexed strategy provides for increased efficacy. In some embodiments, the compositions described herein utilize about 1 gRNA to about 6 gRNAs. In some embodiments, the compositions described herein utilize at least about 1 gRNA. In some embodiments, the compositions described herein utilize at most about 6 gRNAs. In some embodiments, the compositions described herein utilize about 1 gRNA to about 2 gRNAs, about 1 gRNA to about 3 gRNAs, about 1 gRNA to about 4 gRNAs, about 1 gRNA to about 5 gRNAs, about 1 gRNA to about 6 gRNAs, about 2 gRNAs to about 3 gRNAs, about 2 gRNAs to about 4 gRNAs, about 2 gRNAs to about 5 gRNAs, about 2 gRNAs to about 6 gRNAs, about 3 gRNAs to about 4 gRNAs, about 3 gRNAs to about 5 gRNAs, about 3 gRNAs to about 6 gRNAs, about 4 gRNAs to about 5 gRNAs, about 4 gRNAs to about 6 gRNAs, or about 5 gRNAs to about 6 gRNAs. In some embodiments, the compositions described herein utilize about 1 gRNA, about 2 gRNAs, about 3 gRNAs, about 4 gRNAs, about 5 gRNAs, or about 6 gRNAs.

In some embodiments, the gRNA is a synthetic oligonucleotide. In some embodiments, the synthetic nucleotide comprises a modified nucleotide. Modification of the inter-nucleoside linker (i.e. backbone) can be utilized to increase stability or pharmacodynamic properties. For example, inter-nucleoside linker modifications prevent or reduce degradation by cellular nucleases, thus increasing the pharmacokinetics and bioavailability of the gRNA. Generally, a modified inter-nucleoside linker includes any linker other than other than phosphodiester (PO) liners, that covalent), couples two nucleosides together. In some embodiments, the modified inter-nucleoside linker increases the nuclease resistance of the gRNA compared to a phosphodiester linker. For naturally occurring oligonucleotides, the inter-nucleoside linker includes phosphate groups creating a phosphodiester bond between adjacent nucleosides. In some embodiments, the gRNA comprises one or more inter-nucleoside linkers modified from the natural phosphodiester. In some embodiments all of the inter-nucleoside linkers of the gRNA, or contiguous nucleotide sequence thereof; are modified. For example, in some embodiments the inter-nucleoside linkage comprises sulfur (S), such as a phosphorothioate inter-nucleoside linkage.

Modifications to the ribose sugar or nucleobase cat also be utilized herein. Generally, a modified nucleoside includes the introduction of one or more modifications of the sugar moiety or the nucleobase moiety. In some embodiments, the gRNAs, as described, comprise one or more nucleosides comprising a modified sugar moiety, herein the modified sugar moiety is a modification of the sugar moiety when compared to the ribose sugar moiety found in deoxyribose nucleic acid (DNA) and RNA. Numerous nucleosides with modification of the ribose sugar moiety can be utilized, primarily with the aim of improving certain properties of oligonucleotides, such as affinity and/or stability. Such modifications include those where the ribose ring structure is modified. These modifications include replacement with a hexose ring (HNA), a bicyclic ring having a biradical bridge between the C2 and C4 carbons on the ribose ring (e.g. locked nucleic acids (LNA)), or an unlinked ribose ring which typically lacks a bond between the C2 and C3 carbons (e.g. UNA). Other sugar modified nucleosides include, for example, bicyclohexose nucleic acids or tricyclic nucleic acids. Modified nucleosides also include nucleosides where the sugar moiety is replaced with a non-sugar moiety, for example in the case of peptide nucleic acids (PNA), or morpholino nucleic acids.

Sugar modifications also include modifications made by altering the substituent groups on the ribose ring to groups other than hydrogen, or the 2′-OH group naturally found in DNA and RNA nucleosides Substituents may, for example be introduced at the 2′, 3′, 4′ or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity. A 2′ sugar modified nucleoside is a nucleoside that has a substituent other than H or —OH at the 2′ position (2′ substituted nucleoside) or comprises a 2′ linked biradicle, and includes 2′ substituted nucleosides and LNA (2′-4′ biradicle bridged) nucleosides. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. By way of further example, in some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the gRNA comprises one or more modified sugars. In some embodiments, the gRNA comprises only modified sugars. In certain embodiments, the gRNA comprises greater than 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the gRNA comprises both inter-nucleoside linker modifications and nucleoside modifications.

Target specificity can be used in reference to a guide RNA, or a crRNA specific to a target polynucleotide sequence or region (e.g., LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof) and further includes a sequence of nucleotides capable of selectively annealing/hybridizing to a target (sequence or region) of a target polynucleotide (e.g. corresponding to a target), e.g., a target DNA. In some embodiments, a crRNA or the derivative thereof contains a target-specific nucleotide region complementary to a region of the target DNA sequence. In some embodiments, a crRNA or the derivative thereof contains other nucleotide sequences besides a target-specific nucleotide region. In some embodiments, the other nucleotide sequences are from a tracrRNA sequence.

gRNAs are generally supported by a scaffold, wherein a scaffold refers to the portions of gRNA or crRNA molecules comprising sequences which are substantially identical or are highly conserved across natural biological species (e.g. not conferring target specificity). Scaffolds include the tracrRNA segment and the portion of the crRNA segment other than the polynucleotide-targeting guide sequence at or near the 5′ end of the crRNA segment, excluding any unnatural portions comprising sequences not conserved in native crRNAs and tracrRNAs. In some embodiments, the crRNA or tracrRNA comprises a modified sequence. In certain embodiments, the crRNA or tracrRNA comprises at least 1, 2, 3, 4, 5, 10, or 15 modified bases (e.g. a modified native base sequence).

Complementary, as used herein, generally refers to a polynucleotide that includes a nucleotide sequence capable of selectively annealing to an identifying region of a target polynucleotide under certain conditions. As used herein, the term “substantially complementary” and grammatical equivalents is intended to mean a polynucleotide that includes a nucleotide sequence capable of specifically annealing to an identifying region of a target polynucleotide under certain conditions. Annealing refers to the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding in some embodiments, base-stacking and hydrophobic interactions can also contribute to duplex stability. Conditions under which a polynucleotide anneals to complementary or substantially complementary regions of target nucleic acids are well known in the art, e.g., as described in Nucleic Acid Hybridization, A Practical Approach. Hanes and Higgins, eds., IRL Press, Washington, D.C. (1985) and Wetmur and Davidson, Mol. Biol. 31:349 (1968). Annealing conditions will depend upon the particular application and can be routinely determined by persons skilled in the art, without undue experimentation. Hybridization generally refers to process in which two single-stranded polynucleotides bind non-covalently to form a stable double-stranded polynucleotide. A resulting double-stranded polynucleotide is a “hybrid” or “duplex.” In certain instances, 100% sequence identity is not required for hybridization and, in certain embodiments, hybridization occurs at about greater than 70%, 75%, 80% 85%, 90%, or 95% sequence identity. In certain embodiments, sequence identity includes in addition to non-identical nucleobases, sequences comprising insertions and/or deletions.

The nucleic acid of the disclosure, including the RNA (e.g., crRNA, tracrRNA, gRNA) or nucleic acids encoding the RNA, may be produced by standard techniques. For example, polymerase chain reaction (PCR) techniques can be used to obtain an isolated nucleic acid containing a nucleotide sequence described herein, including nucleotide sequences encoding a polypeptide described herein, PCR can be used to amplify specific sequences from DNA as well as RNA, including sequences from total genomic DNA or total cellular RNA. Various PCR methods are described in, for example, PCR Primer: A Laboratory Manual, 2^{nd} edition, Dieffenbach and Dveksler, eds., Cold Spring Harbor Laboratory Press, 2003. Generally, sequence information from the ends of the region of interest or beyond is employed to design oligonucleotide primers that are identical or similar in sequence to opposite strands of the template to be amplified. Venous PCR strategies also are available by which site-specific nucleotide sequence modifications can be introduced into a template nucleic acid.

The isolated nucleic acids also can be chemically synthesized, either as a single nucleic acid (e.g., using automated DNA synthesis in the 3′ to 5′ direction using phosphoramidite technology) or as a series of oligonucleotides. Isolated nucleic acids of the disclosure also can be obtained by mutagenesis of, e.g., a naturally occurring portion crRNA, tracrRNA, RNA-encoding DNA, or of a Cas9-encoding DNA

In certain embodiments, the isolated RNA are synthesized from an expression vector encoding the RNA molecule, as described in detail elsewhere herein.

Nucleic Acids and Vectors

In some embodiments, the composition of the disclosure comprises an isolated nucleic acid encoding one or more elements of the CRISPR-Cas system described herein. For example, in some embodiments, the composition comprises an isolated nucleic acid encoding at least one guide nucleic acid (e.g., gRNA). In some embodiments, the composition comprises an isolated nucleic acid encoding a Cas peptide, or functional fragment or derivative thereof. In some embodiments, the composition comprises an isolated nucleic acid encoding at least one guide nucleic acid (e.g., gRNA) and encoding a Cas peptide, or functional fragment or derivative thereof. In some embodiments, the composition comprises an isolated nucleic acid encoding at least one guide nucleic acid gRNA) and further comprises an isolated nucleic acid encoding a Cas peptide, or functional fragment or derivative thereof.

In some embodiments, the composition comprises at least one isolated nucleic acid encoding a gRNA, where the gRNA is substantially complementary to a target sequences of LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof, as described elsewhere herein. In some embodiments, the composition comprises at least one isolated nucleic acid encoding a gRNA, where the gRNA is complementary to a target sequence hating at least 75%, 80%, 85%, 90%, 95%, 96%, 97%, 98%, or 99% sequence homology to a target sequence described herein.

In some embodiments, the composition comprises at least one isolated nucleic acid encoding a Cas peptide described elsewhere herein, or a functional fragment or derivative thereof. In some embodiments, the composition comprises at least one isolated nucleic acid encoding a Cas peptide having at least 75%, 80%, 85%, 90%, 95%, 96%, 97%, 98%, or 99% amino acid sequence homology with a Cas peptide described elsewhere herein.

The isolated nucleic acid may comprise any type of nucleic acid, including, but not limited to DNA and RNA. For example, in some embodiments, the composition comprises an isolated DNA, including for example, an isolated cDNA, encoding a gRNA or peptide of the disclosure, or functional fragment thereof. In some embodiments, the composition comprises an isolated RNA encoding a peptide of the disclosure, or a functional fragment thereof. The isolated nucleic acids may be synthesized using any method known in the art.

The present disclosure can comprise use of a vector in which the isolated nucleic acid described herein is inserted. The art is replete with suitable vectors that are useful in the present disclosure. Vectors include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-hype or tissue-specific binding), components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors is known in the art and is generally available.

In brief summary, the expression of natural or synthetic nucleic acids encoding an RNA and/or peptide is typically achieved by operably linking a nucleic acid encoding the RNA and/or peptide or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors oldie present disclosure may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the disclosure provides a gene therapy vector.

The isolated nucleic acid of the disclosure can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art.

In some embodiments, lentivirus vectors are used. For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as marine leukemia viruses in that they can transduce non-proliferating cells, such as hepatocytes. They also have the added advantage of low immunogenicity. In some embodiments, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

Further provided are nucleic acids encoding the CRISPR-Cas systems described herein. Provided herein are adeno-associated virus (AAV) vectors comprising nucleic acids encoding the CRISPR-Cas systems described herein. In certain instances, an AAV vector includes to any vector that comprises or derives from components of AAV and is suitable to infect mammalian cells, including human cells, of any of a number of tissue types, such as brain, heart, lung, skeletal muscle, liver, kidney, spleen, or pancreas, whether in vitro or in vivo. In certain instances, an AAV vector includes an AAV type viral particle (or virion) comprising a nucleic acid encoding a protein of interest (e.g. CRISPR-Cas systems described herein). In some embodiments, as further described herein, the AAVs disclosed herein are be derived from various serotypes, including combinations of serotypes (e.g., “pseudotyped” AAV) or from various genomes (e.g., single-stranded or self-complementary). In some embodiments, the AAV vector is a human serotype AAV vector. In such embodiments, a human serotype AAV is derived from any known serotype, e.g., from AAV1, AAV2, AAV4, AAV6, or AAV9. In some embodiments, the serotype is AAV1, AAV2, AAV3, AAV4, AAV5, AAV6, AAV7, AAV8, AAV9, AAV10, AAV11, AAVDJ, or AAVDJ/8.

In some embodiments, the composition includes a vector derived from an adeno-associated virus (AAV). AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

A variety of different AAV capsids have been described and can be used, although AAV which preferentially target the liver and/or deliver genes with high efficiency are particularly desired. The sequences of the AAV8 are available from a variety of databases. While the examples utilize AAV vectors having the same capsid, the capsid of the gene editing vector and the AAV targeting vector are the same AAV capsid. Another suitable AAV is, e.g., rh10 (WO 2003/042397). Still other AAV sources include, e.g., AAV9 (see, for example, U.S. Pat. No. 7,906,111; US 2011-0236353-A1), and/or hu37 (see, e.g., U.S. Pat. No. 7,906,111; US 2011-0236353-A1), AAV1, AAV2, AAV3, AAV4, AAV5, AAV6, AAV6.2, AAV7, AAV8, (U.S. Pat. No. 7,790,449; 7,282,199, WO 2003/042397; WO 2005/033321, WO 2006/110689; U.S. Pat. No. 7,790,449; 7,282,199; 7,588,772). Still other AAV can be selected, optionally taking into consideration tissue preference of the selected AAV capsid.

In some embodiments, AAV vectors disclosed herein include a nucleic acid encoding a CRISPR-Cas systems described herein. In some embodiments, the nucleic acid also includes one or more regulatory sequences allowing expression and, in some embodiments, secretion of the protein of interest, such as e.g., a promoter, enhancer, polyadenylation signal, an internal ribosome entry site (“IRES”), a sequence encoding a protein transduction domain (“PTD”), and the like. Thus, in some embodiments, the nucleic acid comprises a promoter region operably linked to the coding sequence to cause or improve expression of the protein of interest in infected cells. Such a promoter can be ubiquitous, cell- or tissue-specific, strong, weak, regulated, chimeric, etc., for example, to allow efficient and stable production of the protein in the infected tissue. In certain embodiments, the promoter is homologous to the encoded protein, or heterologous, although generally promoters of use in the disclosed methods are functional in human cells. Examples of regulated promoters include, without limitation, Tet on/off element-containing promoters, rapamycin-inducible promoters, tamoxifen-inducible promoters, and metallothionein promoters. In certain embodiments, other promoters used include promoters that are tissue specific for tissues such as kidney, spleen, and pancreas. Examples of ubiquitous promoters include viral promoters, particularly the CAM promoter, the RSV promoter, the SV40 promoter, etc., and cellular promoters such as the phosphoglycerate kinase (PGK) promoter and the b-actin promoter.

In some embodiments, the recombinant AAV vector comprises packaged within an AAV capsid, a nucleic acid, generally containing a 5′ AAV ITR, the expression cassettes described herein and a 3′ AAV ITR. As described herein, in some embodiments, an expression cassette contains regulatory elements for an open reading frame(s) within each expression cassette and the nucleic acid optionally contains additional regulatory elements. The AAV vector, in some embodiments, comprises a full-length AAV 5′ inverted terminal repeat (ITR) and a full-length 3′ ITR. A shortened version of the 5′ ITR, termed ΔITR, has been described in which the D-sequence and terminal resolution site (trs) are deleted. The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription (see, for example, D M McCarty et al, “Self-complementary recombinant adeno-associated virus (scAAV) vectors promote efficient transduction independently of DNA synthesis”, Gene Therapy, (August 2001); see also, for example, U.S. Pat. Nos. 6,596,535; 7,125,717; and 7,456,683). Where a pseudotyped AAV is to be produced, the ITRs are selected from a source which differs from the AAV source of the capsid. For example, in some embodiments, AAV2 ITRs are selected for use with an AAV capsid having a particular efficiency for a selected cellular receptor, target tissue or viral target. In some embodiments, the ITR sequences from AAV2, or the deleted version thereof (ΔITR), are used for convenience and to accelerate regulatory approval (i.e. pseudotyped). In some embodiments, a single-stranded AAV viral vector is used.

Methods for generating and isolating AAV viral vectors suitable for delivery to a subject are known in the art (See, for example, U.S. Pat. No. 7,790,449; 7,282,199; WO 2003/042397; WO 2005/033321, WO 2006/110689; and U.S. Pat. No. 7,588,772 B2, U.S. Pat. Nos. 3,139,941; 5,741,683; 6,057,152; 6,204,059; 6,268,213; 6,491,907; 6,660,514; 6,951,753; 7,094,604; 7,172,893; 7,201,898; 7,229,823; and 7,439,065). In one system, a producer cell line is transiently transfected with a construct that encodes the transgene flanked by ITRs and a construct(s) that encodes rep and cap. In a second system, a packaging cell line that stably supplies rep and cap is transfected (transiently or stably) with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors.

The CRISPR-Cas systems, for instance a Cas9, and/or any of the present RNAs, for instance a guide RNA, can be delivered using adeno associated virus (MV), lentivirus, adenovirus or other viral vector types, or combinations thereof. Cas9 and one or more guide RNAs can be packaged into one or more viral vectors. In some embodiments, the viral vector is delivered to the tissue of interest by, for example, an intramuscular injection, while other times the viral delivery is via intravenous, transdermal, intranasal, oral, mucosal, or other delivery methods. Such delivery can be either via a single dose, or multiple doses. One skilled in the art understands that the actual dosage to be delivered herein can vary greatly depending upon a variety of factors, such as the vector chose, the target cell, organism, or tissue, the general condition of the subject to be treated, the degree of transformation/modification sought, the administration route, the administration mode, the type of transformation/modification sought, etc.

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors may be an indication for some embodiments. The adenovirus vector results in a shatter term expression (e.g., less than about a month) than adeno-associated virus, in some embodiments, may exhibit much longer expression. The particular vector chosen will depend upon the target cell and the condition being treated.

In certain embodiments, the vector also includes conventional control elements which are operably linked to the transgene in a manner which permits its transcription, translation and/or expression in a cell transfected with the plasmid vector or infected with the virus produced by the disclosure. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Additional promoter elements, e.g., enhancers, regulate the frequency of transcriptional initiation, Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

The selection of appropriate promoters can readily be accomplished. In certain aspects, one would use a high expression promoter. One example of a suitable promoter is the immediate early cytomegalovirus (CMV) promoter sequence. This promoter sequence is a strong constitutive promoter sequence capable of driving high levels of expression of any polynucleotide sequence operatively linked thereto. The Rous sarcoma virus (RSV) and MMT promoters may also be used. Certain proteins can be expressed using their native promoter. Other elements that can enhance expression can also be included such as an enhancer or a system that results in high levels of expression such as a tat gene and tar element. This cassette can then be inserted into a vector, e.g., a plasmid vector such as, pUC19, pUC118, pBR322, or other known plasmid vectors, that includes, for example, an E. coli origin of replication.

Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatinine kinase promoter. Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In certain embodiments, HIV-1 expression dependent CRISPR vectors comprise a minimal HIV-1 Tat-inducible promoter LTR −80/+66. A “minimal” promoter or “truncated” promoter or “functional fragment” of a promoter includes all essential elements of a promoter for transcriptional activation of, for example, a nucleic acid sequence operably linked or under control of the minimal promoter. In one embodiment, a truncated HIV long terminal repeat (LTR) promoter comprises at least a core region, a trans activation response element (TAR) or combinations thereof, of a HIV LTR, promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In same embodiments, the vector of the present disclosure comprises one or more enhancers to boost transcription of the gene present within the vector.

In order to assess the expression of the nucleic acid and/or peptide, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tel et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, heads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in sire or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant nucleic acid sequence in the host cell, a variety of assays may be performed. Such assays include, for example, “molecular biological” assays well known to those of skill in the art, such as Southern and Northern blotting, RT-PCR and PCR, “biochemical” assays, such as detecting the presence or absence of a particular peptide, e.g., by immunological means (ELISAs and Western blots) or by assays described herein to identify agents falling within the scope of the disclosure.

In certain embodiments, the composition comprises a cell genetically modified to express one or more isolated nucleic acids and/or peptides described herein. For example, the cell may be transfected or transformed with one or more vectors comprising an isolated nucleic acid sequence encoding a gRNA and/or a Cas peptide. The cell can be the subject's cells or they can be haplotype matched or a cell line. The cells can be irradiated to prevent replication. In some embodiments, the cells are human leukocyte antigen (HLA)-matched, autologous, cell lines, or combinations thereof. In other embodiments the cells can be a stem cell. For example, an embryonic stem cell or an artificial pluripotent stem cell (induced pluripotent stem cell (iPS cell)). Embryonic stem cells (ES cells) and artificial pluripotent stem cells (induced pluripotent stem cell, iPS cells) have been established from many animal species, including humans. These types of pluripotent stem cells would be the most useful source of cells for regenerative medicine because these cells are capable of differentiation into almost all of the organs by appropriate induction of their differentiation, with retaining their ability of actively dividing while maintaining their pluripotency, iPS cells, in particular, can be established from self-derived somatic cells, and therefore are not likely to cause ethical and social issues, in comparison with ES cells which are produced by destruction of embryos. Further, iPS cells, which are a self-derived cell, make it possible to avoid rejection reactions, which are the biggest obstacle to regenerative medicine or transplantation therapy.

Pharmaceutical Compositions

The compositions described herein are suitable for use in a variety of drug delivery systems described above. Additionally, in order to enhance the in vivo serum half-life of the administered compound, the compositions may be encapsulated, introduced into the lumen of liposomes, prepared as a colloid, or other conventional techniques may be employed which provide an extended serum half-life of the compositions. A variety of methods are available for preparing liposomes, as described in, e.g., Szoka, el al., U.S. Pat. Nos. 4,235,871, 4,501,728 and 4,837,028 each of which is incorporated herein by reference. Furthermore, one may administer the drug in a targeted drug delivery system, for example, in a liposome coated with a tissue-specific antibody. The liposomes will be targeted to and taken up selectively by the organ.

The present disclosure also provides pharmaceutical compositions comprising one or more of the compositions described herein. Formulations may be employed in admixtures with conventional excipients, i.e., pharmaceutically acceptable organic or inorganic carrier substances suitable for administration to the wound or treatment site. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other analgesic agents.

Administration of the compositions of this disclosure may be carried out, for example, by parenteral, by intravenous, intratumoral, subcutaneous, intramuscular, or intraperitoneal injection, or by infusion or by any other acceptable systemic method. Formulations for administration of the compositions include those suitable for rectal, nasal, oral, topical (including buccal and sublingual), vaginal or parenteral (including subcutaneous, intramuscular, intravenous and intradermal) administration. The formulations may conveniently be presented in unit dosage form, e.g. tablets and sustained release capsules, and may be prepared by any methods well known in the art of pharmacy.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials Other “additional ingredients” that may be included in the pharmaceutical compositions of the disclosure are known in the an and described, for example in Genaro, ed. (1985, Remington's Pharmaceutical Sciences, Mack Publishing Co., Easton, Pa.), which is incorporated herein by reference.

The composition of the disclosure may comprise a preservative from about 0.005% to 2.0% by total weight of the composition. The preservative is used to prevent spoilage in the case of exposure to contaminants in the environment. Examples of preservatives useful in accordance with the disclosure included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

In an embodiment, the composition includes an anti-oxidant and a chelating agent that inhibits the degradation of one or more components of the composition. Preferred antioxidants for some compounds are BHT. BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While Riff and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension the composition of the disclosure in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as Liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid.

Combination Therapies

In certain embodiments, the gene-editing compositions embodied herein are administered to a patient in combination with one or more other anti-viral agents or therapeutics. The term “combination therapy”, as used herein, refers to those situations in which two or more different pharmaceutical agents are administered in overlapping regimens so that the subject is simultaneously exposed to both agents. When used in combination therapy, two or more different gents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

Examples include any molecules that are used for the treatment of a virus and include agents which alleviate any symptoms associated with the virus, for example, anti pyretic agents, anti-inflammatory agents, chemotherapeutic agents, and the like. An antiviral agent includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof.

Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs. The therapeutic agents may be administered under a metronomic regimen. As used herein, “metronomic” therapy refers to the administration of continuous low-doses of a therapeutic agent.

The compositions can be administered in conjunction with (e.g., before, simultaneously or following) one or more therapies. For example, in certain embodiments, the method comprises administration of a composition of the disclosure in conjunction with an additional anti-viral therapy, cancer therapy and the like.

Methods of Treatment

The present disclosure provides a method of treating or preventing a retrovirus, e.g., HTLV infection. In some embodiments, the method comprises administering to a subject in need thereof, an effective amount of a composition comprising at least one of a guide nucleic acid and a Cas peptide, or functional fragment or derivative thereof. In some embodiments, the method comprises administering a composition comprising an isolated nucleic acid encoding at least one of the guide nucleic acid and a Cas peptide, or functional fragment or derivative thereof. In certain embodiments, the method comprises administering a composition described herein to a subject diagnosed with a retrovirus, e.g., HTLV infection, at risk for developing a retrovirus, e.g., HTLV infection and the like.

Provided herein, in certain embodiments, are methods of modifying and/or excising and/or editing a LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof, in the genome of a cell (e.g. host cell) using the CRISPR-Cas systems or compositions described herein. Generally, of modifying and/or excising and/or editing the target sequences in the genome of a cell (e.g. host cell) comprises contacting a cell, or providing to the cell, a CRISPR-Cas system or composition targeting one or more regions in the LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof. In some embodiments, the methods comprise removing or excising a sequence from a genome of the cell. In some embodiments, the methods result in excising at least or about 100, 200, 300, 400, 500, 600, 700, 800, 900, 1000, 2000, 3000, 4000, 5000, 6000, 7000, 8000, 9000, or more than 9000 base pairs of the LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof, in a host cell.

Dosage, toxicity and therapeutic efficacy of the present compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. The Cas9/gRNA compositions that exhibit high therapeutic indices are preferred. While Cas9/gRNA compositions that exhibit toxic side effects may be used, care should be taken to design a delivery system that targets such compositions to the site of affected tissue in order to minimize potential damage to uninfected cells and, thereby, reduce side effects.

The data obtained from the cell culture assays and animal studies can be used in formulating a range of dosage for use in humans. The dosage of such compositions lies preferably within a range of circulating concentrations that include the ED₅₀ with little or no toxicity. The dosage may vary within this range depending upon the dosage form employed and the route of administration utilized. For any composition used in the method of the disclosure, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a composition (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the disclosure can include a single treatment or a series of treatments.

The gRNA expression cassette can be delivered to a subject by methods known in the art. In some aspects, the Cas may be a fragment wherein the active domains of the Cas molecule are included, thereby cutting down on the size of the molecule. Thus, the, Cas/gRNA molecules can be used clinically, similar to the approaches taken by current gene therapy.

In some embodiments, the method comprises genetically modifying a cell to express a guide nucleic acid and/or Cas peptide. For example, in some embodiments, the method comprises contacting a cell with an isolated nucleic acid encoding the guide nucleic acid and/or Cas peptide.

In some embodiments, for viral vector-mediated delivery, a dose comprises at least 1×10⁵ particles to about 1×10¹³ particles. In some embodiments the delivery is via an adenovirus, such as a single dose containing at least 1×10⁵ particles (also referred to as particle units, pu) of adenoviral vector. In some embodiments, the dose is at least about 1×10⁶ particles (for example, about 1×10⁶-1×10¹² particles), at least about 1×10⁷ particles, at least about 1×10⁸ particles (e.g., about 1×10⁸-1×10¹¹ particles or about 1×10⁸-1×10¹² particles), at least about 1×10⁸ particles (e.g., about 1×10⁹-1×10¹⁰ particles or about 1×10⁹-1×10¹² particles), or at least about 1×10¹⁰ particles (e.g., about 1×10-1×10¹² particles) of the adenoviral vector. Alternatively, the dose comprises no more than about 1×10¹⁴ particles, no more than about 1×10¹³ particles, no more than about 1×10¹² particles, no more than about 1×10¹¹ particles, and no more than about 1×10¹⁰ particles (e.g., no more than about 1×10⁹ particles). Thus, in, some embodiments, the dose contains a single dose of adenoviral vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10⁹ pu, about 4×10⁹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹² pu, about 2×10¹² pu, or about 4×10¹² pu of adenoviral vector. In some embodiments, the adenovirus is delivered via multiple doses.

In some embodiments, the delivery is via an AAV. A therapeutically effective dosage for in vivo delivery of the AAV to a human is believed to be in the range of from about 20 to about 50 ml of saline solution containing from about 1×10¹⁰ to about 1×10¹⁰ functional AAV/ml solution. The dosage can be adjusted to balance therapeutic benefit against any side effects. In some embodiments, the AAV dose is generally in the range of concentrations of from about 1×10⁵ to 1×10⁵⁰ genres AAV, from about 1×10⁸ to 1×10²⁰ genomes AAV, from about 1×10¹⁰ to about 1×10⁵⁰ genomes, or about 1×10¹¹ to about 1×10¹⁶ genomes AAV. In some embodiments, a human dosage is about 1×10¹³ genres AAV. In some embodiments, such concentrations are delivered in from about 0.001 ml to about 100 ml, about 0.05 to about 50 ml, or about 10 to about 25 ml of a carrier solution. Other effective dosages can be readily established by one of ordinary skill in the art through routine trials establishing dose response curves (see, for example, U.S. Pat. No. 8,404,658).

In some embodiments, the cell is genetically modified in vivo in the subject in whom the therapy is intended. In certain aspects, for in viva, delivery the nucleic acid is injected directly into the subject. For example, in some embodiments, the nucleic acid is delivered at the site where the composition is required. In vivo nucleic acid transfer techniques include, but is not limited to, transfection with viral vectors such as adenovirus, Herpes simplex I virus, adeno-associated virus), lipid-based systems (useful lipids for lipid-mediated transfer of the gene are DOTMA, DOPE and DC-Chol, for example), naked DNA, and transposon-based expression systems. Exemplary gene therapy protocols see Anderson et al., Science 256:808-813 (1992). See also WO 93/25673 and the references cited therein. In certain embodiments, the method comprises administering of RNA, for example mRNA, directly into the subject (see for example, Zangi et al., 2013 Nature Biotechnology, 31, 898-907).

For ex vivo treatment, an isolated cell is modified in an ex vivo or in vitro environment. In some embodiments, the cell is autologous to a subject to whom the therapy is intended. Alternatively, the cell can be allogeneic, syngeneic, or xenogeneic with respect to the subject. The modified cells may then be administered to the subject directly.

One skilled in the art recognizes that different methods of deli very may be utilized to administer an isolated nucleic acid into a cell. Examples include: (1) methods utilizing physical means, such as electroporation (electricity), a gene gun (physical force) or applying large volumes of a liquid (pressure); and (2) methods wherein the nucleic acid or vector is complexed to another entity, such as a liposome, aggregated protein or transporter molecule.

The amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present disclosure (for instance, the cost associated with synthesis). One skilled in the art can easily make any necessary adjustments in accordance with the exigencies of the particular situation.

Genetically modified cells may also contain a suicide gene i.e., a gene which encodes a product that can be used to destroy the cell. In many gene therapy situations, it is desirable to be able to express a gene for therapeutic purposes in a host, cell but also to have the capacity to destroy the host cell at will. The therapeutic agent can be linked to a suicide gene, whose expression is not activated in the absence of an activator compound. When death of the cell in which both the agent and the suicide gene have been introduced is desired, the activator compound is administered to the cell thereby activating expression of the suicide gene and killing the cell. Examples of suicide gene/prodrug combinations which may be used are herpes simplex virus-thymidine kinase (HSV-tk) and ganciclovir, acyclovir; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

### EXAMPLES

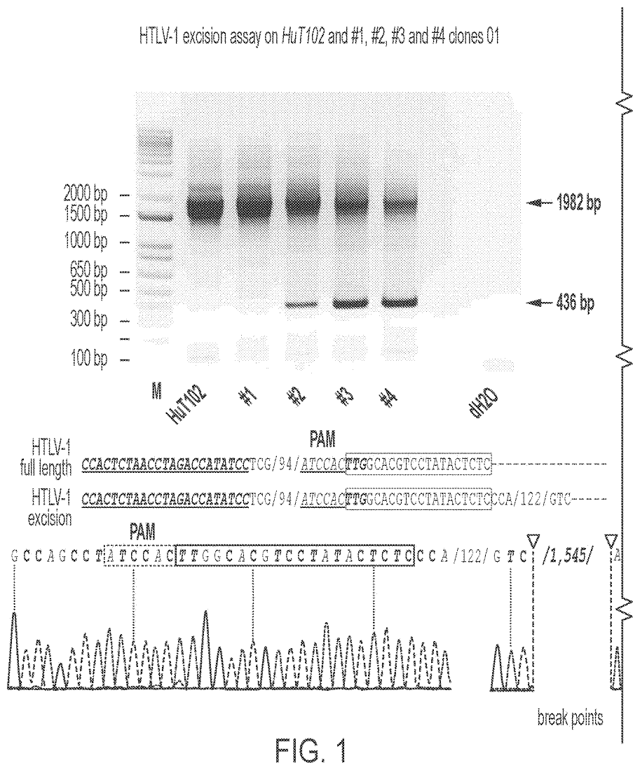

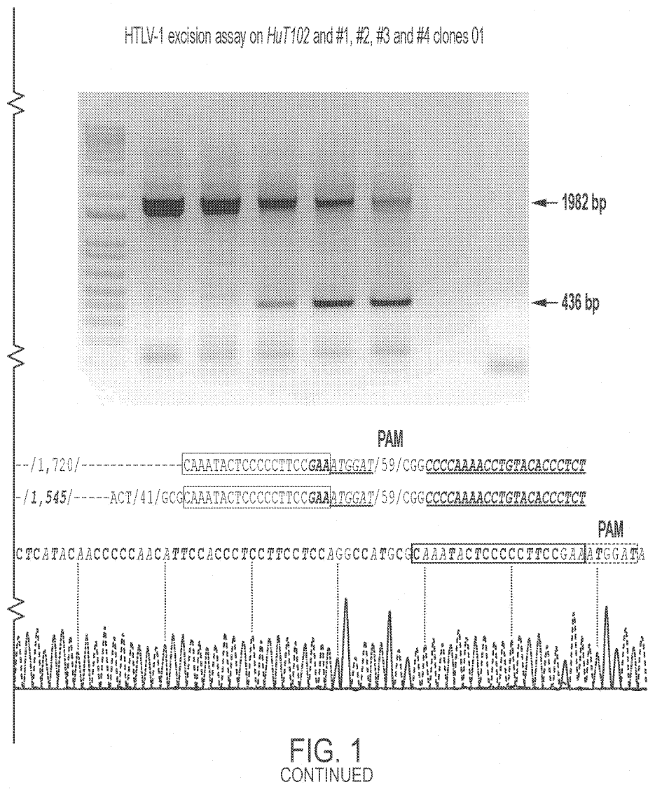

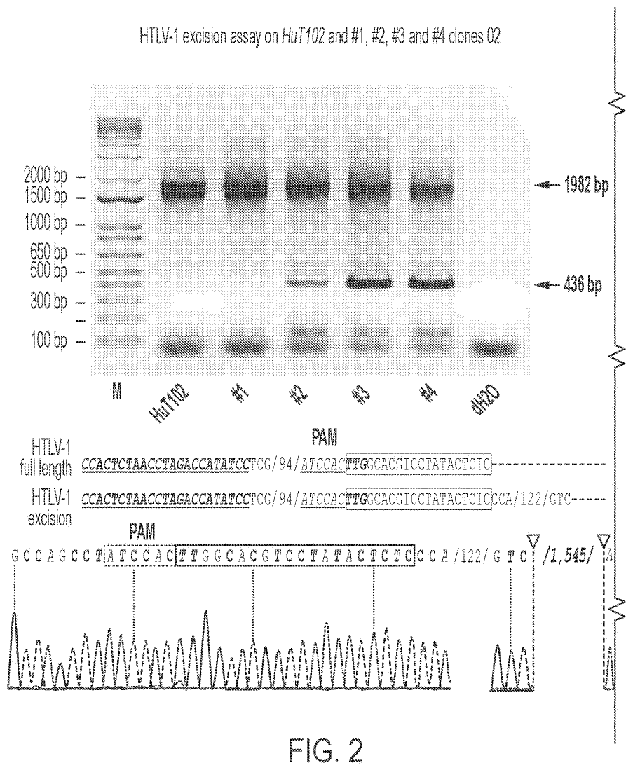

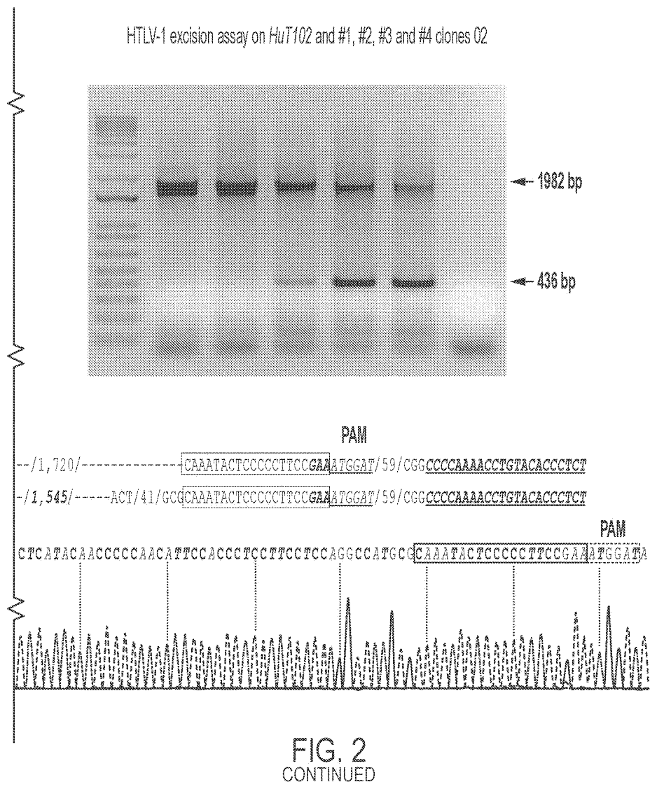

Cells infected with HTLV-1 were subjected to CRISPR/Cas endonuclease targeted to HTLV genes. FIG. 1 and FIG. 2 are results from are blots demonstrating the excision of HTLV-1.

FIG. 1:

HTLV-1 full length sequence (SEQ ID NO: 1):
CCACTCTAACCTAGACCATATCCTCG/94/ATCCAC
TTGGCACGTCCTATACTCTC . . . /1,720/ . . . C
AAATACTCCCCCTTCCGAAATGGAT/59/CGGCCCCAAAACC
TGTACACCCTCT
HTLV-1 Excision (SEQ ID NO: 2):
CCACTCTAACCTAGACCATATCCTCG/94/ATCCACTTGGCA
CGTCCTATACTCTCCCA/122/GTC. . . /
1,545/ . . . ACT/
41/GCGCAAATACTCCCCCTTCCGAAATGGA
T/59/CGGCCCCAAAACCTGTACACCCTCT

### Guide Nucleic Acid Sequences

SEQ ID NO: 3
ATCCACTTGGCACGTCCTATACTCTC
SEQ ID NO. 4
CAAATACTCCCCCTTCCGAAATGGAT
SEQ ID NO: 5
ATCCACTTGGCACGTCCTATACTCTCCCA
SEQ ID NO: 6
GCGCAAATACTCCCCCTTCCGAAATGGAT

Bolded nucleotides are the PAM sequences.

FIG. 2:

HTLV-1 full length sequence (SEQ ID NO: 1):
CCACTCTAACCTAGACCATATCCTCG/94/ATCCAC
TTGGCACGTCCTATACTCTC . . . /1,720/ . . . C
AAATACTCCCCCTTCCGAAATGGAT/59/CGGCCCCAAAACC
TGTACACCCTCT
HTLV-1 Excision (SEQ ID NO: 2):
CCACTCTAACCTAGACCATATCCTCG/94/ATCCACTTGGCA
CGTCCTATACTCTCCCA/122/GTC. . . /
1,545/ . . . ACT/
41/GCGCAAATACTCCCCCTTCCGAAATGGA
T/59/CGGCCCCAAAACCTGTACACCCTCT

### Guide Nucleic Acid Sequences

SEQ ID NO: 3
ATCCACTTGGCACGTCCTATACTCTC
SEQ ID NO. 4
CAAATACTCCCCCTTCCGAAATGGAT
SEQ ID NO: 5
ATCCACTTGGCACGTCCTATACTCTCCCA
SEQ ID NO: 6
GCGCAAATACTCCCCCTTCCGAAATGGAT

Bolded nucleotides are the PAM sequences.

1. A gene editing complex comprising an isolated nucleic acid sequence encoding a clustered regularly interspaced short palindromic repeats (CRISPR)-associated endonuclease and at least one guide RNA (gRNA), the gRNA being complementary to a target nucleic acid sequence in a Human T cell leukemia virus type 1 or 2 (HTLV-1 or -2) genome, comprising LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof.

2. (canceled)

3. (canceled)

4. (canceled)

5. The gene-editing complex of claim 1, wherein the CRISPR-associated endonuclease is a Type I, Type II, or Type III Cas endonuclease.

6. The gene-editing complex of claim 5, wherein the CRISPR-associated endonuclease is a Cas9 endonuclease, a Cas12 endonuclease, a CasX endonuclease, a CasΦ endonuclease or variants thereof.

7. The gene-editing complex of claim 6, wherein the CRISPR-associated endonuclease is a Cas9 nuclease or variants thereof.

8. The gene-editing complex of claim 7, wherein the Cas9 nuclease is a Staphylococcus aureus Cas9 nuclease.

9. The gene-editing complex of claim 7, wherein the Cas9 variant comprises one or more point mutations, relative to wildtype Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A.

10. The gene-editing complex of claim 1, wherein the CRISPR-associated endonuclease is optimized for expression in a human cell.

11. The gene-editing complex of claim 1, wherein the guide nucleic acid sequence comprises a sequence comprising at least about 90% sequence identity to any one of SEQ ID NOS: 3-6, or a complement of any one of SEQ ID NOS: 3-6.

12. The gene-editing complex of claim 1, wherein the guide nucleic acid sequence comprises a sequence of any one of SEQ ID NOS: 3-6, or a complement of any one of SEQ ID NOS: 3-6 or combinations thereof.

13. (canceled)

14. (canceled)

15. The gene-editing complex of claim 1, wherein the target nucleic acid sequences comprise a sequence comprising at least about 90% sequence identity to at least five consecutive nucleotides of SEQ ID NOS: 1 or 2, or a complement of at least five consecutive nucleotides of SEQ ID NOS: 1 or 2.

16. The gene-editing complex of claim 1, wherein the target nucleic acid sequence comprises at least five consecutive nucleotides of SEQ ID NOS: 1 or 2, or at least five consecutive nucleotides complementary to SEQ ID NOS: 1 or 2, or combinations thereof.

17. The gene-editing complex of claim 1, wherein the isolated nucleic acid sequences are included in at least one expression vector selected from the group consisting of: a lentiviral vector, an adenovirus vector, an adeno-associated virus vector, a vesicular stomatitis virus (VSV) vector, a pox virus vector, and a retroviral vector.

18. (canceled)

19. (canceled)

20. (canceled)

21. (canceled)

22. (canceled)

23. A composition comprising:
a) a Clustered Regularly Interspaced Short Palindromic Repeat (CRISPR)-associated endonuclease or a nucleic acid sequence encoding the CRISPR-associated endonuclease;
b) one or more guide nucleic acids, wherein the guide nucleic acids comprise nucleotide sequences substantially complementary to a target sequence comprising: LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof.

24. The composition of claim 23, wherein the CRISPR-associated endonuclease is a Type I, Type II, or Type III Cas endonuclease.

25. The composition of claim 24, wherein the CRISPR-associated endonuclease is a Cas9 endonuclease, a Cas 12 endonuclease, a Cas 13 endonuclease, a CasX endonuclease, a CasΦ endonuclease or variants thereof.

26. The composition of claim 25, wherein a Cas9 variant comprises a human-optimized Cas9; a nickase mutant Cas9; saCas9; enhanced-fidelity SaCas9 (efSaCas9); SpCas9(K855a); SpCas9(K810A/K1003 A/r 1060 A); SpCas9(K848A/K1003A/R1060A); SpCas9 N497A, R661A, Q695A, Q926A; SpCas9 N497A, R661A, Q695A, Q926A, D1135E; SpCas9 N497A, R661A, Q695A, Q926A L169A; SpCas9 N497A, R661A, Q695A, Q926A Y450A; SpCas9 N497A, R661A, Q695A, Q926A M495A; SpCas9 N497A, R661A, Q695A, Q926A M694A; SpCas9 N497A, R661A, Q695A, Q926AH698A; SpCas9 N497A, R661A, Q695A, Q926A, D1135E, LI 69 A; SpCas9 N497A, R661A, Q695A, Q926A, D1135E, Y450A; SpCas9 N497A, R661A, Q695A, Q926A, D1135E, M495A; SpCas9 N497A, R661A, Q695A, Q926A, D1135E, M694A; SpCas9 N497A, R661A, Q695A, Q926A, D1135E, M698A; SpCas9 R661A, Q695A, Q926A; SpCas9 R661A, Q695A, Q926A, D1135E; SpCas9 R661A, Q695A, Q926A, L169A; SpCas9 R661A, Q695A, Q926A Y450A; SpCas9 R661A, Q695A, Q926AM495A; SpCas9 R661A, Q695A, Q926A M694A; SpCas9 R661A, Q695A, Q926A H698A; SpCas9 R661A, Q695A, Q926A D1135E L169A; SpCas9 R661A, Q695A, Q926A D1135E Y450A; SpCas9 R661A, Q695A, Q926A D1135E M495A; or SpCas9 R661A, Q695A, Q926A, D1135E or M694A.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. A method of treating a subject infected with a Human T cell leukemia virus (HTLV) comprising:
(i) administering to the subject an effective amount of a gene editing complex comprising an isolated nucleic acid sequence encoding a clustered regularly interspaced short palindromic repeats (CRISPR)-associated endonuclease and at least one guide RNA (gRNA), the gRNA being complementary to a target nucleic acid sequence in a Human T cell leukemia virus type 1 or 2 (HTLV-1 or -2) genome, comprising LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof;
or
(ii) administering to the subject an effective amount of a composition comprising:
a) a Clustered Regularly Interspaced Short Palindromic Repeat (CRISPR)-associated endonuclease or a nucleic acid sequence encoding the CRISPR-associated endonuclease;
b) one or more guide nucleic acids, wherein the guide nucleic acids comprise nucleotide sequences substantially complementary to a target sequence comprising: LTR nucleic acid sequences, Gag nucleic acid sequences, Pol nucleic acid sequences, Pro nucleic acid sequences, Env nucleic acid sequences, pX region nucleic acid sequences, HBZ nucleic acid sequences, APH-2 nucleic acid sequences, Tax-1 nucleic acid sequences, Tax-2 nucleic acid sequences or combinations thereof;
whereby the genome between the two target regions is excised.

49. The method of claim 48, wherein the isolated nucleic acid sequences are included in at least one expression vector selected from the group consisting of: a lentiviral vector, an adenovirus vector, an adeno-associated virus vector, a vesicular stomatitis virus (VSV) vector, a pox virus vector, and a retroviral vector.


SEQUENCE LISTING (9 sequences)

SEQ ID NO: 1 - DNA, 26 bp, Human T-cell leukemia virus type 1
  source 1..26: mol_type = genomic DNA
ccactctaacctagaccatatcctcg

SEQ ID NO: 2 - DNA, 26 bp, Human T-cell leukemia virus type 1
  source 1..26: mol_type = genomic DNA
ccactctaacctagaccatatcctcg

SEQ ID NO: 3 - DNA, 26 bp, Human T-cell leukemia virus type 1
  source 1..26: mol_type = genomic DNA
atccacttggcacgtcctatactctc

SEQ ID NO: 4 - DNA, 26 bp, Human T-cell leukemia virus type 1
  source 1..26: mol_type = genomic DNA
caaatactcccccttccgaaatggat

SEQ ID NO: 5 - DNA, 29 bp, Human T-cell leukemia virus type 1
  source 1..29: mol_type = genomic DNA
atccacttggcacgtcctatactctccca

SEQ ID NO: 6 - DNA, 29 bp, Human T-cell leukemia virus type 1
  source 1..29: mol_type = genomic DNA
gcgcaaatactcccccttccgaaatggat

SEQ ID NO: 7 - DNA, 25 bp, Human T-cell leukemia virus type 1
  source 1..25: mol_type = genomic DNA
cggccccaaaacctgtacaccctct

SEQ ID NO: 8 - DNA, 37 bp, Human T-cell leukemia virus type 1
  source 1..37: mol_type = genomic DNA
gccagcctatccacttggcacgtcctatactctccca

SEQ ID NO: 9 - DNA, 74 bp, Human T-cell leukemia virus type 1
  source 1..74: mol_type = genomic DNA
actcatacaacccccaacattccaccctccttcctccaggccatgcgcaaatactcccccttccgaaatggata